SEQUENCE LISTING (15 sequences)

SEQ ID NO: 1 - protein, 250 aa, Homo sapiens [sequence omitted]

SEQ ID NO: 2 - protein, 7 aa, synthetic construct
  REGION 1..7: note = TB006 Heavy Chain CDR1 Polypeptide Sequence
GYSFTNY

SEQ ID NO: 3 - protein, 8 aa, synthetic construct
  REGION 1..8: note = TB006 Heavy Chain CDR2 Polypeptide Sequence
IYPGSGNT

SEQ ID NO: 4 - protein, 9 aa, synthetic construct
  REGION 1..9: note = TB006 Heavy Chain CDR3 Polypeptide Sequence
STAPGGFDV

SEQ ID NO: 5 - protein, 11 aa, synthetic construct
  REGION 1..11: note = TB006 Light Chain CDR1 Polypeptide Sequence
KSLLHSDGITY

SEQ ID NO: 6 - skipped

SEQ ID NO: 7 - protein, 9 aa, synthetic construct
  REGION 1..9: note = TB006 Light Chain CDR3 Polypeptide Sequence
AQMLEFPLT

SEQ ID NO: 8 - protein, 116 aa, synthetic construct
  REGION 1..116: note = TB006 Heavy Chain Variable Domain Polypeptide Sequence
QVQLVQSGAEVKKPGASVKVSCKASGYSFTNYYMHWVRQAPGQRLEWMGWIYPGSGNTNYNEKFQGRVTITADTSASTAYMELSSLRSEDTAVYYCSTAPGGFDVWGQGTTVTVSS

SEQ ID NO: 9 - protein, 112 aa, synthetic construct
  REGION 1..112: note = TB006 Light Chain Variable Domain Polypeptide Sequence
DIVMTQTPLSLSVTPGQPASISCKSSKSLLHSDGITYLYWYLQKPGQSPQLLIYRMSNLASGVPDRFSGSGSGTDFTLKISRVEAEDVGVYYCAQMLEFPLTFGQGTKLEIK

SEQ ID NO: 10 - protein, 466 aa, synthetic construct
  REGION 1..466: note = TB006 Heavy Chain Polypeptide Sequence
MDPKGSLSWRILLFLSLAFELSYGQVQLVQSGAEVKKPGASVKVSCKASGYSFTNYYMHWVRQAPGQRLEWMGWIYPGSGNTNYNEKFQGRVTITADTSASTAYMELSSLRSEDTAVYYCSTAPGGFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 239 aa, synthetic construct
  REGION 1..239: note = TB006 Light Chain Polypeptide Sequence
METDTLLLWVLLLWVPGSTGDIVMTQTPLSLSVTPGQPASISCKSSKSLLHSDGITYLYWYLQKPGQSPQLLIYRMSNLASGVPDRFSGSGSGTDFTLKISRVEAEDVGVYYCAQMLEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 357 bp, synthetic construct
  misc_feature 1..357: note = TB006 Heavy Chain Variable Domain Nucleic Acid Sequence
  source 1..357: mol_type = other DNA
cagattcaattggttcagagcggctcagaactgaaaaagcctggagccagcgttaaagtgagctgtaaagccagtggatacacattcaccacttacgtcatgtcctgggttagacaggcccctggacaggggctggaatggatgggctggataaacactcactctggagtgcccacatacgccgacgactttacaggcaggttcgtgttttccctagatactagtgtgaatactgcatacttgcaaatctcttctctgaaagcagaggataccgccgtgtacttttgcacccgcgatgggaatgatggggatgccatggacaactggggccaggggacgactgtgacagtgtcttca

SEQ ID NO: 13 - DNA, 336 bp, synthetic construct
  misc_feature 1..336: note = TB006 Light Chain Variable Domain Nucleic Acid Sequence
  source 1..336: mol_type = other DNA
gatatagtgatgacccaaacacctttgtccctgtcagtcaccccaggccaacccgcatctatctcatgcaaatcctcaaaaagtcttttgcactctgacgggataacatatctctactggtatcttcaaaaacctggtcagtccccccagctcttgatctatagaatgagtaaccttgcctctggtgtccccgaccggtttagtggaagcgggtctgggaccgacttcaccctcaagattagtcgcgtcgaagccgaagatgtgggagtgtattactgtgcccagatgctcgaatttcccctgacatttggtcaaggaactaaattggagattaag

SEQ ID NO: 14 - DNA, 1341 bp, synthetic construct
  misc_feature 1..1341: note = TB006 Heavy Chain Nucleic Acid Sequence
  source 1..1341: mol_type = other DNA
cagattcaattggttcagagcggctcagaactgaaaaagcctggagccagcgttaaagtgagctgtaaagccagtggatacacattcaccacttacgtcatgtcctgggttagacaggcccctggacaggggctggaatggatgggctggataaacactcactctggagtgcccacatacgccgacgactttacaggcaggttcgtgttttccctagatactagtgtgaatactgcatacttgcaaatctcttctctgaaagcagaggataccgccgtgtacttttgcacccgcgatgggaatgatggggatgccatggacaactggggccaggggacgactgtgacagtgtcttcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 15 - DNA, 660 bp, synthetic construct
  misc_feature 1..660: note = TB006 Light Chain Nucleic Acid Sequence
  source 1..660: mol_type = other DNA
gatatagtgatgacccaaacacctttgtccctgtcagtcaccccaggccaacccgcatctatctcatgcaaatcctcaaaaagtcttttgcactctgacgggataacatatctctactggtatcttcaaaaacctggtcagtccccccagctcttgatctatagaatgagtaaccttgcctctggtgtccccgaccggtttagtggaagcgggtctgggaccgacttcaccctcaagattagtcgcgtcgaagccgaagatgtgggagtgtattactgtgcccagatgctcgaatttcccctgacatttggtcaaggaactaaattggagattaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

(19) United States
(12) Patent Application Publication
(10) Pub. No.: US 2024/0158512 A1
(43) Pub. Date: May 16, 2024

(54) ANTI-GAL3 ANTIBODY FORMULATIONS AND METHODS OF USE THEREOF

(71) Applicant: TrueBinding, Inc., Foster City, CA (US)

(72) Inventors: Dongxu Sun, Palo Alto, CA (US); Yan He, Fremont, CA (US); Fan Chen, Sunnyvale, CA (US); Apurva Chandalia, Union City, CA (US); Ksenya Shchors, San Mateo, CA (US)

(21) Appl. No.: 18/492,607

(22) Filed: Oct. 23, 2023

Related U.S. Application Data
(63) Continuation of application No. PCT/US2022/026005, filed on Apr. 22, 2022.
(60) Provisional application No. 63/179,879, filed on Apr. 26, 2021.

(51) Int. Cl. C07K 16/28; A61K 47/02; A61K 47/20; A61K 47/22; A61K 47/26; A61P 25/28

(52) U.S. Cl. CPC C07K 16/2851; A61K 47/02; A61K 47/20; A61K 47/22; A61K 47/26; A61P 25/28

(57) ABSTRACT

Disclosed herein are pharmaceutical antibody formulations for the treatment of a disease. The pharmaceutical antibody formulations comprise an antibody that binds to Galectin-3 (Gal3) and one or more excipients, diluents, salts, buffers, and the like. Also disclosed herein are sterile vials comprising these pharmaceutical antibody formulations, optionally in a concentrated form that is diluted prior to administration. The pharmaceutical antibody formulations are used to treat a disease such as a neurodegenerative disease, proteopathy, and/or inflammation associated with the aforementioned diseases.

### CROSS-REFERENCE TO RELATED APPLICATIONS

This application is a bypass continuation in the United States of PCT International Application No. PCT/US2022/026005, filed Apr. 22, 2022, which claims the benefit of priority of U.S. Provisional Patent Application No. 63/179,879, filed Apr. 26, 2021, which are hereby expressly incorporated by reference in their entirety.

### REFERENCE TO SEQUENCE LISTING

The present application is being filed along with a Sequence Listing in electronic format. The Sequence Listing is provided as a file entitled IMMUT028C1SEQLIST.XML, which was created and last modified on Oct. 20, 2023, which is 22,276 bytes in size. The information in the electronic Sequence Listing is hereby incorporated by reference in its entirety.

### FIELD

Aspects of the present disclosure relate generally to pharmaceutical antibody formulations comprising antibodies that bind to Galectin-3 (Gal3) and one or more excipients, diluents, salts, buffers, and the like. These pharmaceutical antibody formulations are used to treat a disease such as a neurodegenerative disease, proteopathy, and/or inflammation associated with the aforementioned diseases.

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

Blockade of Gal3 with antibodies have beneficial effects such as reducing inflammation and promoting regeneration of various cell types. There is a present need for improved formulations of said antibodies, such as those optimized for administration in a mammal, optionally a human.

### SUMMARY

Disclosed herein are embodiments of pharmaceutical antibody formulations. In some embodiments, the pharmaceutical antibody formulations comprise a therapeutically effective amount of an antibody. In some embodiments, the antibody is and/or includes an anti-Gal3 antibody. In some embodiments, the antibody comprises a heavy chain CDR1 (HCDR1) having the sequence of SEQ ID NO: 2, a heavy chain CDR2 (HCDR2) having the sequence of SEQ ID NO: 3, a heavy chain CDR3 (HCDR3) having the sequence of SEQ ID NO: 4, a light chain CDR1 (LCDR1) having the sequence of SEQ ID NO: 5, a light chain CDR2 (LCDR2) having the sequence of SEQ ID NO: 6; and a light chain CDR3 (LCDR3) having the sequence of SEQ ID NO: 7. In some embodiments, the antibody is TB006 (4A11.H3L1, IMT006a, IMT006-5). In some embodiments, the pharmaceutical antibody formulation further comprises one or more of histidine, methionine, NaCl, or polysorbate. In some embodiments, the pharmaceutical antibody formulation comprises histidine, methionine, NaCl, and polysorbate. In some embodiments, the pharmaceutical antibody formulation is at a pH between 5.3 and 6.3.

Also disclosed herein are embodiments of pharmaceutical antibody formulations. In some embodiments, the pharmaceutical antibody formulations comprise a therapeutically effective amount of an antibody. In some embodiments, the antibody is an anti-Gal3 antibody. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg, or any amount as a unit dose within a range defined by any two of the aforementioned amounts as a unit dose. In some embodiments, the antibody is present as an amount as a unit dose of 70 mg. In some embodiments, the antibody is present as an amount as a unit dose of 75 mg. In some embodiments, the antibody is present as an amount as a unit dose of 140 mg. In some embodiments, the antibody is present as an amount as a unit dose of 200 mg. In some embodiments, the antibody is present as an amount as a unit dose of 420 mg. In some embodiments, the antibody is present as an amount as a unit dose of 450 mg. In some embodiments, the antibody is present as an amount as a unit dose of 700 mg. In some embodiments, the antibody is present as an amount as a unit dose of 1500 mg. In some embodiments, the antibody is present as an amount as a unit dose of 2100 mg. In some embodiments, the antibody is present as an amount as a unit dose of 3750 mg. In some embodiments, the antibody is present as an amount as a unit dose of 7500 mg. In some embodiments, the pharmaceutical antibody formulation further comprise L-histidine present at 20 mM, methionine present at 5 mM, NaCl present at 100 mM, and polysorbate 80 present at 0.02%. In some embodiments, the pharmaceutical antibody formulation comprises a pH of about 5.8.

Also disclosed herein are embodiments of sterile vials comprising a pharmaceutical antibody formulation. In some embodiments, the pharmaceutical antibody formulation comprises a therapeutically effective amount of an antibody. In some embodiments, the antibody is an anti-Gal3 antibody. In some embodiments, the antibody comprises an HCDR1 having the sequence of SEQ ID NO; 2, an HCDR2 having the sequence of SEQ ID NO: 3, an HCDR3 having the sequence of SEQ ID NO; 4, an LCDR1 having the sequence of SEQ ID NO: 5, an LCDR2 having the sequence of SEQ ID NO: 6; and an LCDR3 having the sequence of SEQ ID NO: 7.

Also disclosed herein are embodiments of methods of treating Alzheimer's disease. In some embodiments, the methods comprise administering any one of the pharmaceutical antibody formulations disclosed herein to a subject in need of Alzheimer's disease treatment.

Also disclosed herein are embodiments of pharmaceutical antibody formulations. In some embodiments, the pharmaceutical antibody formulation comprises a therapeutically effective amount of an antibody. In some embodiments, the antibody is an anti-Gal3 antibody. In some embodiments, the antibody comprises a HCDR1 having the sequence of SEQ ID NO: 2, a HCDR2 having the sequence of SEQ ID NO: 3, a HCDR3 having the sequence of SEQ ID NO: 4, a LCDR1 having the sequence of SEQ ID NO: 5, a LCDR2 having the sequence of SEQ ID NO: 6; and a LCDR3 having the sequence of SEQ ID NO: 7. In some embodiments, each CDR can have up to 1, 2, 3, 4, or 5 amino acids changed from the recited sequence. In some embodiments, the pharmaceutical antibody formulation further comprises one or more of histidine, methionine, NaCl, or polysorbate. In some embodiments, the pharmaceutical antibody formulation comprises histidine, methionine, NaCl, and polysorbate. In some embodiments, the pharmaceutical antibody formulation is at a pH between 5.3 and 6.3. In some embodiments, the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg. In some embodiments, the antibody is present at an amount as a unit dose of 70 mg. In some embodiments, the antibody is present at an amount as a unit dose of 75 mg. In some embodiments, the antibody is present at an amount as a unit dose of 140 mg. In some embodiments, the antibody is present at an amount as a unit dose of 200 mg. In some embodiments, the antibody is present at an amount as a unit dose of 420 mg. In some embodiments, the antibody is present at an amount as a unit dose of 450 mg. In some embodiments, the antibody is present at an amount as a unit dose of 700 mg. In some embodiments, the antibody is present at an amount as a unit dose of 1500 mg. In some embodiments, the antibody is present at an amount as a unit dose of 2100 mg. In some embodiments, the antibody is present at an amount as a unit dose of 3750 mg. In some embodiments, the antibody is present at an amount as a unit dose of 5000 mg. In some embodiments, the antibody is present at an amount as a unit dose of 7500 mg.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts the peptide sequence for Gal3.

FIG. 2A depicts exemplary heavy chain and light chain complementarity-determining regions (CDRs) for anti-Gal3 antibodies disclosed herein. In some embodiments, any of the pharmaceutical antibody formulations may comprise an antibody with one or more CDRs provided herein.

FIG. 2B depicts exemplary heavy chain and light chain variable regions (VH and VL respectively), heavy chain (HC) and light chain (LC) polypeptide sequences. In some embodiments, any of the pharmaceutical antibody formulations may comprise an antibody with one or more VH, VL, HC, and/or LC provided herein.

FIG. 2C depicts exemplary nucleic acid sequences that encode for anti-Gal3 antibody VH, VL, HC, and/or LC polypeptide sequences. In some embodiments, any of the pharmaceutical antibody formulations may comprise an antibody that is encoded by any of the nucleic acid sequences provided herein.

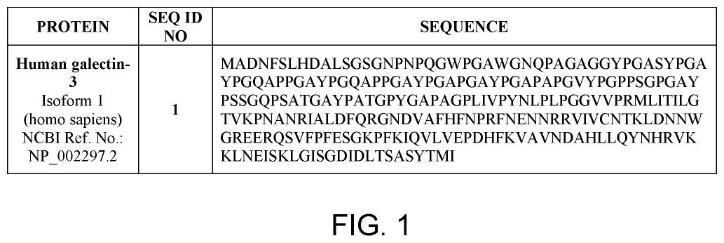

### DETAILED DESCRIPTION OF THE DISCLOSURE

Some embodiments provided herein relate to pharmaceutical antibody formulations. In some embodiments, the pharmaceutical antibody formulations comprise a therapeutically effective amount of an antibody, where the antibody is an anti-Gal3 antibody. In some embodiments, the pharmaceutical antibody formulations further comprise one or more excipients, diluents, salts, buffers, and the like. In some embodiments, the pharmaceutical antibody formulations are at a certain pH.

Some embodiments provided herein relate to pharmaceutical antibody formulations comprising a therapeutically effective amount of an antibody, histidine, methionine, NaCl, and polysorbate, where the formulation is at a pH between 5.3 and 6.3. In some embodiments, the histidine is L-histidine. In some embodiments, the polysorbate is polysorbate 80. In some embodiments, the pharmaceutical antibody formulations further comprise sucrose or mannitol, or both.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg. In some embodiments, the antibody is present as an amount as a unit dose of 70 mg. In some embodiments, the antibody is present at an amount as a unit dose of 75 mg. In some embodiments, the antibody is present as an amount as a unit dose of 140 mg. In some embodiments, the antibody is present as an amount as a unit dose of 200 mg. In some embodiments, the antibody is present as an amount as a unit dose of 420 mg. In some embodiments, the antibody is present at an amount as a unit dose of 450 mg. In some embodiments, the antibody is present as an amount as a unit dose of 700 mg. In some embodiments, the antibody is present at an amount as a unit dose of 1500 mg. In some embodiments, the antibody is present as an amount as a unit dose of 2100 mg. In some embodiments, the antibody is present at an amount as a unit dose of 3750 mg. In some embodiments, the antibody is present as an amount as a unit dose of 5000 mg. In some embodiments, the antibody is present at an amount as a unit dose of 7500 mg.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises a VH region having a sequence at least 80%, 85%, 90%, 95%, 99%, or 100% identical to that of SEQ ID NO: 8. In some embodiments, the antibody comprises a VL region having a sequence at least 80%, 85%, 90%, 95%, 99%, or 100% identical to that of SEQ ID NO: 9. In some embodiments, the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises a VH region having a sequence at least 80%, 85%, 90%, 95%, 99%, or 100% identical to that of SEQ ID NO: 8. In some embodiments, the antibody comprises a VL region having a sequence at least 80%, 85%, 90%, 95%, 99%, or 100% identical to that of SEQ ID NO: 9. In some embodiments, the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9. In some embodiments, the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg. In some embodiments, the antibody is present as an amount as a unit dose of 70 mg. In some embodiments, the antibody is present at an amount as a unit dose of 75 mg. In some embodiments, the antibody is present as an amount as a unit dose of 140 mg. In some embodiments, the antibody is present as an amount as a unit dose of 200 mg. In some embodiments, the antibody is present as an amount as a unit dose of 420 mg. In some embodiments, the antibody is present at an amount as a unit dose of 450 mg. In some embodiments, the antibody is present as an amount as a unit dose of 700 mg. In some embodiments, the antibody is present at an amount as a unit dose of 1500 mg. In some embodiments, the antibody is present as an amount as a unit dose of 2100 mg. In some embodiments, the antibody is present at an amount as a unit dose of 3750 mg. In some embodiments, the antibody is present as an amount as a unit dose of 5000 mg. In some embodiments, the antibody is present at an amount as a unit dose of 7500 mg.

Some embodiments provided herein relate to pharmaceutical antibody formulations comprising a therapeutically effective amount of an antibody, histidine present at a concentration of 20 mM, methionine present at a concentration of 5 mM, NaCl present at a concentration of 100 mM, polysorbate present at a concentration of 0.02%, and where the formulation is at a pH of about 5.8. In some embodiments, the histidine is L-histidine. In some embodiments, the polysorbate is polysorbate 80. In some embodiments, the pharmaceutical antibody formulations further comprise sucrose or mannitol, or both. In some embodiments, the sucrose is present at a concentration of 2-5%. In some embodiments, the mannitol is present at a concentration of 2-5%. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises a VH region having a sequence at least 80%, 85%, 90%, 95%, 99%, or 100% identical to that of SEQ ID NO: 8. In some embodiments, the antibody comprises a VL region having a sequence at least 80%, 85%, 90%, 95%, 99%, or 100% identical to that of SEQ ID NO: 9. In some embodiments, the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9.

Some embodiments provided herein relate to sterile vials comprising any one of the pharmaceutical antibody formulations disclosed herein. In some embodiments, the sterile vials can contain a certain volume. In some embodiments, the sterile vials contain a certain volume of the pharmaceutical antibody formulations. In some embodiments, the sterile vials contain a certain amount of antibody. In some embodiments, the sterile vials contain a concentrated form of any one of the pharmaceutical antibody formulations disclosed herein.

Some embodiments provided herein relate to methods of treating Alzheimer's disease. In some embodiments, the methods comprise administering any one of the pharmaceutical antibody formulations disclosed herein to a subject in need of Alzheimer's disease treatment. In some embodiments, the subject is a mammal. In some embodiments, the subject is a human.

Galectin-3 (Gal3, GAL3) plays an important role in cell proliferation, adhesion, differentiation, angiogenesis, and apoptosis. This activity is, at least in part, due to immunomodulatory properties and binding affinity towards other immune regulatory proteins, signaling proteins, and other cell surface markers. Gal3 functions by distinct N-terminal and C-terminal domains. The N-terminal domain (isoform 1: amino acids 1-111) comprise a tandem repeat domain (TRD, isoform 1: amino acids 36-109) and is largely responsible for oligomerization of Gal3. The C-terminal domain (isoform 1: amino acids 112-250) comprise a carbohydrate-recognition-binding domain (CRD), which binds to β-galactosides.

Galectin-3 (Gal3) has been implicated to have immunomodulatory activity. An example of this is the interaction between Gal3 and T-cell immunoglobulin and mucin-domain containing-3 (TIM-3), which causes suppression of immune responses such as T cell activation and may enable cancer cells to evade immune clearance. This phenomenon and methods to inhibit the same are explored in WO 2019/023247, hereby expressly incorporated by reference in its entirety. Anti-Gal3 antibodies and methods of use thereof have also been explored, for example, in PCT Publication WO 2020/160156, hereby expressly incorporated by reference in its entirety.

Provided herein are anti-Gal3 antibody formulations, some of which can be for the treatment of various diseases. Formulations intended for a certain disease may vary in term of qualities such as amount and concentration of antibody, amount and concentration of excipients, number of doses, and frequency and duration of administration.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Unless otherwise specified, the complementarity defining regions disclosed herein follow the IMGT definition. In some embodiments, the CDRs can instead by Kabat, Chothia, or other definitions accepted by those of skill in the art.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some embodiments, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In some non-limiting examples, an effective amount or effective dose of a composition or compound may relate to the amount or dose that provides a significant, measurable, or sufficient therapeutic effect towards the treatment of a neurological disease or proteopathy, such as Alzheimer's disease, or a symptom thereof. In some embodiments, the effective amount or effective dose of a composition or compound may treat, ameliorate, or prevent the progression of memory loss, dementia, disorientation, or any other symptom of Alzheimer's disease.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g. PD1/PDL1 or CTLA4 blockade therapy).

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), tris(hydroxymethyl)aminomethane (Tris), citric acid, ascorbic acid, acetic acid, salts, phosphates, citrates, acetates, succinates, chlorides, bicarbonates, borates, sulfates, sodium chloride, sodium bicarbonate, sodium phosphate, sodium borate, sodium citrate, potassium chloride, potassium phosphate, magnesium sulfate sugars, dextrose, dextran 40, fructose, mannose, lactose, trehalose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, poloxamer, poloxamer 188, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

As used herein, the term “neurological disorder” refers to a disease affecting the central and/or peripheral nervous system of a patient. A neurological disorder has a physical cause, such as external or internal mechanical trauma (e.g. stroke or concussion), biological trauma (e.g. infection), chemical trauma (e.g. toxins or drugs), aging and age-related senescence, genetics, and many other causes. Some neurological disorders are caused by the effect or accumulation of mutated or misfolded proteins. These diseases may involve the death of neurons or other cell types associated with the nervous system. Non-limiting examples of neurological disorders include inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. Some neurological disorders can also be categorized as proteopathies.

As used herein, the term “proteopathy” refers to a disease which is caused by abnormal folding or accumulation of proteins. An abnormal protein may gain a toxic function, or lose their normal function. It is possible that misfolded proteins can induce the misfolding of otherwise normally folded proteins, resulting in an amplification of the disease (e.g. prion disease). Some non-limiting examples of proteopathies include Alzheimer's disease, cerebral 0-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art.

As used herein, the term “Alzheimer's disease” refers to the neurodegenerative disease that causes loss of memory, dementia, and eventual death. The cause of Alzheimer's disease is multifactorial and is not fully understood. A common hypothesized cause is the aggregation and accumulation of amyloid beta (A3) peptides in the brain, resulting in neuronal degeneration and inflammation. Alzheimer's disease is currently uncurable, and currently available therapeutics, such as cholinesterase inhibitors and NDMA receptor antagonists, have minimal effect on the progression of the disease and/or only treat secondary symptoms of the disease. Applicant has previously shown that anti-Gal3 antibodies have an effect on reducing A3 oligomerization and is potentially an Alzheimer's disease therapeutic.

The term “% w/w” or “% wt/wt” means a percentage expressed in terms of the weight of the ingredient or agent over the total weight of the composition multiplied by 100.

### Exemplary Pharmaceutical Anti-Gal3 Antibody Formulations

Disclosed herein are pharmaceutical antibody formulations. These pharmaceutical antibody formulations can be used for therapeutic applications. In some embodiments, the pharmaceutical antibody formulations comprise a therapeutically effective amount of an antibody, such as an anti-Gal3 antibody. In some embodiments, the antibody is any of the anti-Gal3 antibodies disclosed herein or otherwise known in the art, such as those described in WO 2020/160156. The pharmaceutical antibody formulations may also comprise one or more excipients, diluents, salts, buffers, and the like, which confer desirable properties to the formulation, such as improved stability, reduction in aggregation, and modulation of isotonicity and pH. It is envisioned that one or more excipients, diluents, salts, buffers, and the like generally known in the art can be used in the pharmaceutical antibody formulations disclosed herein and/or can be used as an acceptable substitute for any of the excipients, diluents, salts, buffers, and the like used in the pharmaceutical antibody formulations disclosed herein, and determining an optimal formulation of excipients, diluents, salts, buffers, and the like is within the ability of one skilled in the art. The inclusion of one or more excipients, diluents, salts, buffers, and the like may be adjusted for the treatment of a certain disease, such as a neurological disorder or proteopathy, such as Alzheimer's disease, or inflammation associated with said diseases, and/or optimized to improve the stability of the pharmaceutical antibody formulations under storage.

Disclosed in some embodiments are pharmaceutical antibody formulations comprising an anti-Gal3 antibody and one or more excipients. The one or more excipients may be used to improve stability of the anti-Gal3 antibody under storage conditions and/or improve biocompatibility when administered to a subject. The one or more excipients may comprise small molecules, amino acids, peptides, proteins, nucleic acids, DNA, RNA, lipids, ionic compounds, salts, carbohydrates, sugars, sugar alcohols, acids, bases, surfactants, detergents, or other excipients known in the art. In some embodiments, the pharmaceutical antibody formulations are at a specific pH that improves stability of the anti-Gal3 antibody under storage conditions and/or improve biocompatibility when administered to a subject. In some embodiments, the one or more excipients are used to adjust the pH to the desired level. In some embodiments, the pH of the pharmaceutical antibody formulations are adjusted after addition of the one or more excipients to the desired pH (e.g. by addition of a compatible acid or base, such as HCl, H₂SO₄, acetic acid, citric acid, phosphates, NaOH, KOH, etc.). The pharmaceutical antibody formulations may be acidic, basic, or neutral.

In some embodiments of the pharmaceutical antibody formulations disclosed herein comprising an anti-Gal3 antibody and one or more excipients, the one or more excipients comprise one or more amino acids, one or more salts, one or more surfactants, or any combination thereof. In some embodiments, the one or more amino acids may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or any combination thereof. The one or more amino acids used as excipients may be L-stereoisomers or D-stereoisomers. In some embodiments, the one or more amino acids may be in the form of small peptides, such as dipeptides, tripeptides, tetrapeptides, or more. Some embodiments of the pharmaceutical antibody formulations comprise histidine or methionine, or both. In some embodiments, the histidine or methionine, or both, are L-stereoisomers or D-stereoisomers. Other amino acids, either as substitutes of histidine or methionine, or both, or in addition to histidine or methionine, or both, are also envisioned in some embodiments, depending on the desired properties conferred to the formulations, such as improved stability, pH adjustment, and compatibility to the intended subject. Some non-limiting embodiments of pharmaceutical antibody formulations may comprise 1) histidine and methionine, 2) histidine and any one or more other amino acids, 3) methionine and any one or more other amino acids, 4) one or more amino acids other than histidine and methionine (e.g. one or more of arginine, glycine, or glutamate), or 5) histidine, methionine, and one or more other amino acids (e.g. one or more of arginine, glycine, or glutamate).

In some embodiments of the pharmaceutical antibody formulations disclosed herein comprising an anti-Gal3 antibody and one or more excipients, including the ones comprising one or more amino acids as disclosed herein, the one or more excipients comprise one or more salts. In some embodiments, the one or more salts may comprise salts conventionally used as excipients. In some embodiments, the one or more salts may comprise chloride salts, phosphate salts, carbonate salts, bicarbonate salts, citrate salts, ascorbate salts, acetate salts, succinate salts, Tris salts, borate salts, sulfate salts, ammonia salts, metal salts, sodium salts, potassium salts, calcium salts, magnesium salts, organic salts, amino acid salts, nucleic acid salts, aromatic salts, low solubility salts, and the like, including any disclosed throughout the present disclosure. The purpose of using one or more salts as an excipient includes but is not limited to improving stability and reducing aggregation of an antibody, equalizing ionic charges for other components in the formulation, adjusting solubility of other components in the formulation, adjusting pH and isotonicity, and improving biocompatibility for administration to a subject. Exemplary pharmaceutical antibody formulations disclosed herein comprise NaCl. However, alternative salts may also be used, either instead of NaCl or in addition to NaCl, including but not limited to those provided herein, such as other chloride salts, other sodium salts, ascorbate salts, acetate salts, phosphate salts, citrate salts, Tris salts, or succinate salts, or those otherwise known in the art.

In some embodiments of the pharmaceutical antibody formulations disclosed herein comprising an anti-Gal3 antibody and one or more excipients, including the ones comprising one or more amino acids and/or one or more salts as disclosed herein, the one or more excipients comprise one or more surfactants. In some embodiments, the one or more surfactants may comprise surfactants conventionally used as excipients. In some embodiments, the one or more surfactants may include polysorbate, polysorbate 20, polysorbate 40, polysorbate 60, polysorbate 80, oils, poloxamers, poloxamer 188, polyglycosides, cetyl alcohol, cocamides, stearates, laurates, nonoxynols, octoxynols, or other surfactants generally known in the art and used as excipients, including any disclosed throughout the present disclosure. In some embodiments, the surfactants may also act as wetting agents, detergents, or emulsifying agents, depending on the specific surfactant and the intended purpose. The purpose of these surfactants in the pharmaceutical antibody formulations may include but are not limited to improving the solubility of the antibody or the other excipients, improving stability of the antibody, and preventing aggregation of the antibody. Exemplary pharmaceutical antibody formulations disclosed herein comprise a polysorbate. In some embodiments, the polysorbate may be polysorbate 20, polysorbate 40, polysorbate 60, or polysorbate 80, or any combination thereof. In some embodiments, the polysorbate is polysorbate 80. However, alternative surfactants may also be used, either instead of the polysorbate, or in addition to the polysorbate, including but not limited to those provided herein, such as poloxamer 188, or those otherwise known in the art.

In some embodiments of the pharmaceutical antibody formulations disclosed herein comprising an anti-Gal3 antibody and one or more excipients, including the ones comprising one or more amino acids, one or more salts, and/or one or more surfactants disclosed herein, the one or more excipients may also comprise one or more sugars or sugar alcohols. In some embodiments, the one or more sugars or sugar alcohols include those conventionally used as excipients. In some embodiments, the sugars include but are not limited to erythrose, arabinose, ribose, deoxyribose, xylose, galactose, glucose (dextrose), fructose, isomaltose, lactose, maltose, sucrose, trehalose, maltodextrin, chitosan, dextrin, dextran, dextran 40, cellulose, or starch, or other sugars generally known in the art and used as excipients, including any disclosed throughout the present disclosure. In some embodiments, the sugar alcohols include but are not limited to glycerol, erythritol, arabitol, xylitol, ribitol, deoxyribitol, mannitol, sorbitol, galactitol, isomalt, maltitol, or lactitol, or other sugar alcohols generally known in the art and used as excipients, including any disclosed throughout the present disclosure. The purpose of these sugars and sugar alcohols in the pharmaceutical antibody formulations may include but are not limited to improving the stability and preventing aggregation of the antibody. Exemplary pharmaceutical antibody formulations disclosed herein may comprise a sugar or a sugar alcohol, or both. In some embodiments, exemplary pharmaceutical antibody formulations comprise sucrose and/or mannitol. However, alternative sugars and sugar alcohols may be used, either instead of the sucrose and/or mannitol, or in addition to the sucrose and/or mannitol, including but not limited to those provided herein, such as sorbitol, trehalose, dextrose, dextran, or dextran 40. In some embodiments, formulations that are intended for subcutaneous use comprise any one or more of the sugars or sugar alcohols disclosed herein, including sucrose and/or mannitol. In some embodiments, formulations that are intended for intravenous use might not comprise any one or more of the sugars or sugar alcohols disclosed herein, including sucrose and/or mannitol.

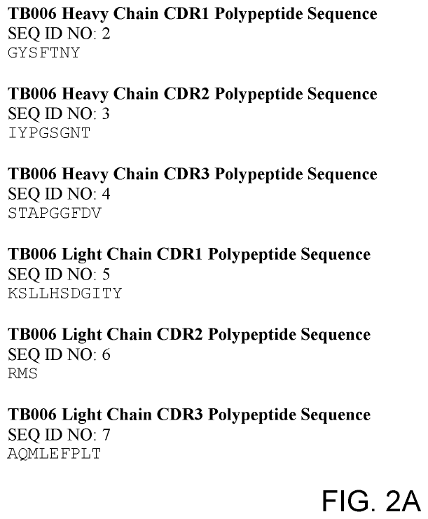

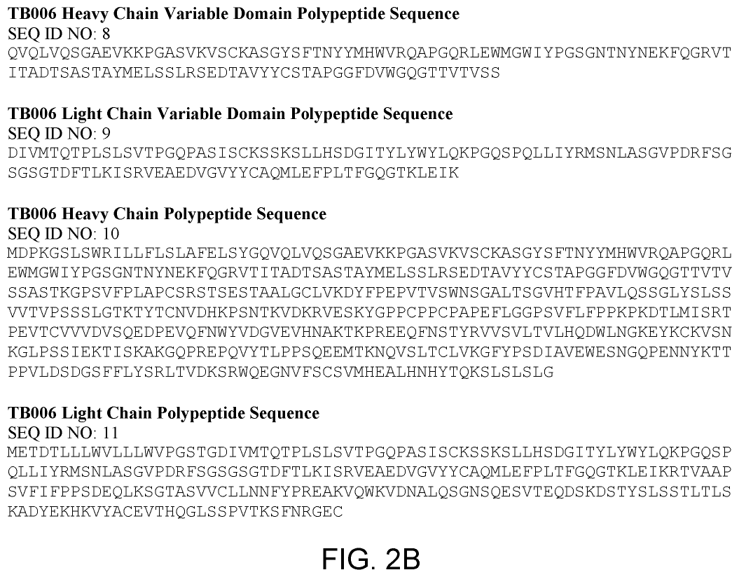

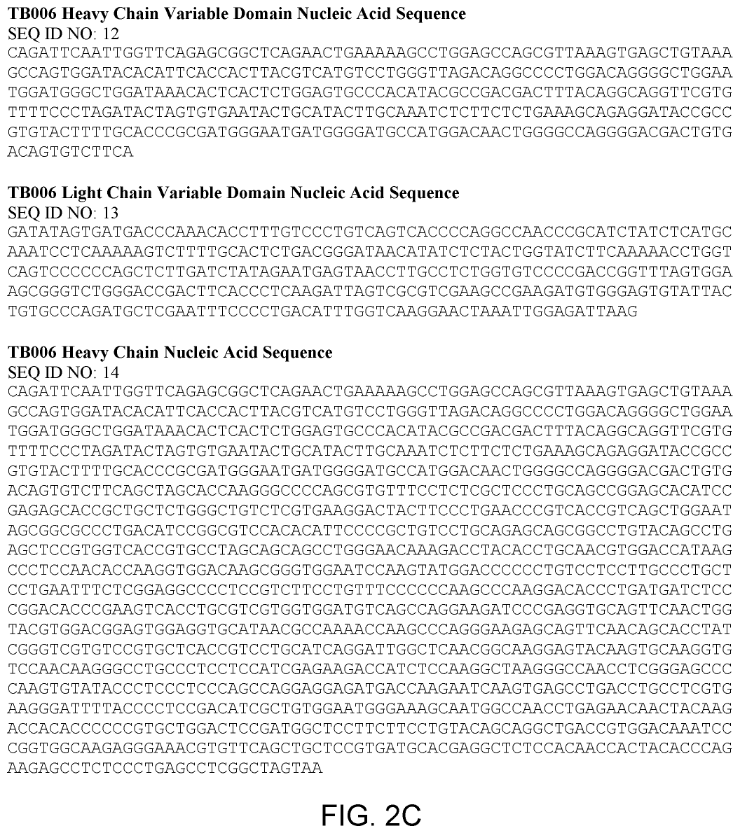

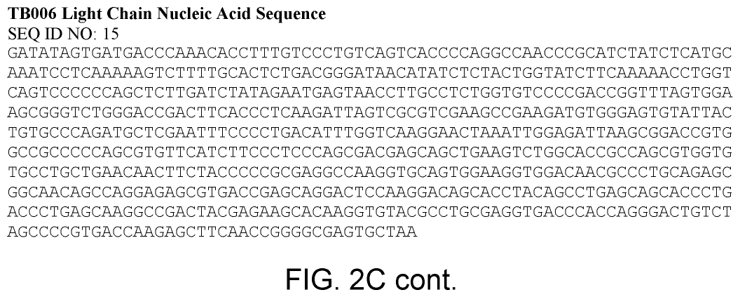

In some embodiments, the pharmaceutical antibody formulations comprise an anti-Gal3 antibody. In some embodiments, the anti-Gal3 antibody is any one of the anti-Gal3 antibodies disclosed herein, or otherwise known in the art, such as those disclosed in WO 2020/160156. The anti-Gal3 antibody may be a full length antibody, an Fab fragment, an F(ab′)₂ fragment, an scFv, an sdAb, a monovalent fragment, or any other modified antibody known in the art, including bispecific, trispecific, and other multi-specific variants. The anti-Gal3 antibody will generally comprise complementarity-determining regions (CDRs). In some embodiments, the anti-Gal3 antibody may comprise a heavy chain CDR1 (HCDR1), heavy chain CDR2 (HCDR2), heavy chain CDR3 (HCDR3) and/or light chain CDR1 (LCDR1), light chain CDR2 (LCDR2), or light chain CDR3 (LCDR3), or any combination thereof. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, exemplary CDR sequences are depicted in FIG. 2A. In some embodiments, each CDR can have up to 1, 2, 3, 4, or 5 amino acids changed from the recited sequence. In some embodiments, each CDR can have a sequence at least 80%, 85%, 90%, 95%, 99%, or 100% identical to those depicted in FIG. 2A. In some embodiments, the anti-Gal3 antibody comprises a VH having a sequence at least 80%, 85%, 90%, 95%, 99%, or 100% identical to that of SEQ ID NO: 8. In some embodiments, the anti-Gal3 antibody comprises having a sequence at least 80%, 85%, 90%, 95%, 99%, or 100% identical to that of SEQ ID NO: 9. The pharmaceutical antibody formulations may comprise one or more excipients. In some embodiments, the one or more excipients are present in amounts that are optimized for a certain disease or disorder, to improve stability and/or biocompatibility when administered to a subject. In some embodiments, the one or more excipients may be present in a concentration that is, is about, is not more than, or is not less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mM, or any concentration within a range defined by any two of the aforementioned concentrations. The one or more excipients may also be present in a concentration that is, is about, is not more than, or is not less than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any combination within a range within any two of the aforementioned concentrations. In some embodiments, the pharmaceutical antibody formulations further comprise one or more amino acids, one or more salts, or one or more surfactants (which may make up the one or more excipients). The one or more amino acids, one or more salts, and one or more surfactants may be any one of the amino acids, salts, and surfactants disclosed herein. In some embodiments, the one or more amino acids are present at 10 to 50 mM or about 10 to about 50 mM. In some embodiments, the one or more amino acids are present at 20 mM or about 20 mM. In some embodiments, the one or more salts are present at 50 to 150 mM or about 50 to about 150 mM. In some embodiments, the one or more salts are present at 100 mM or about 100 mM. In some embodiments, the one or more surfactants are present at 0.01% to 0.04% or about 0.01% to about 0.04%. In some embodiments, the one or more surfactants are present at 0.02% or about 0.02%. In some embodiments, the formulation is at a pH between 5.3 and 6.3. In some embodiments, the formulation is at a pH of 5.8 or about 5.8.

In some embodiments, the pharmaceutical antibody formulations comprise an anti-Gal3 antibody. In some embodiments, the anti-Gal3 antibody is any one of the anti-Gal3 antibodies disclosed herein, or otherwise known in the art, such as those disclosed in WO 2020/160156. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, exemplary CDR sequences are depicted in FIG. 2A. In some embodiments, each CDR can have up to 1, 2, 3, 4, or 5 amino acids changed from the recited sequence. In some embodiments, the anti-Gal3 antibody comprises a VH having a sequence at least 80%, 85%, 90%, 95%, 99%, or 100% identical to that of SEQ ID NO: 8. In some embodiments, the anti-Gal3 antibody comprises having a sequence at least 80%, 85%, 90%, 95%, 99%, or 100% identical to that of SEQ ID NO: 9. In some embodiments, the pharmaceutical antibody formulations further comprise one or more amino acids, one or more salts, or one or more surfactants. In some embodiments, the one or more amino acids comprise histidine and/or methionine, the one or more salts comprise sodium chloride (NaCl), the one or more surfactants comprise a polysorbate, or any combination thereof. In some embodiments, the pharmaceutical antibody formulations comprise histidine, methionine, NaCl, or polysorbate, or any combination thereof, including all of histidine, methionine, NaCl, and polysorbate. In some embodiments, the histidine may be L-histidine. In some embodiments, the L-histidine is present at 10 to 50 mM or about 10 to about 50 mM, or any amount or concentration envisioned herein. In some embodiments, the L-histidine is present at 20 mM or about 20 mM. In some embodiments, the methionine is present at 2 to 10 mM or about 2 to about 10 mM, or any amount or concentration envisioned herein. In some embodiments, the methionine is present at 5 mM or about 5 mM. In some embodiments, the NaCl is present at 50 to 150 mM or about 50 to about 150 mM, or any amount or concentration envisioned herein. In some embodiments, the NaCl is present at 100 mM or about 100 mM. In some embodiments, the polysorbate comprises polysorbate-20, polysorbate-40, polysorbate-60, polysorbate-80, or any combination thereof. In some embodiments, the polysorbate is or comprises polysorbate-80. In some embodiments, the polysorbate-80 is present at 0.01% to 0.04% or about 0.01% to about 0.04%, or any amount or concentration envisioned herein. In some embodiments, the polysorbate-80 is present at 0.02% or about 0.02%. In some embodiments, the formulation is at a pH between 5.3 and 6.3. In some embodiments, the formulation is at a pH of 5.8 or about 5.8.

In some embodiments, the formulation includes one or more of: Polysorbate 80, Polysorbate 20, Poloxamer 188, Mannitol, Sorbitol, Sucrose, Trehalose, Dextrose, Dextran 40, NaCl, Arginine, Glycine, Methionine, Ascorbic acid, NaOAc, Phosphate, Citrate, Acetate, Tris, Succinate, Histidine.

In some embodiments, pharmaceutical antibody formulations are provided. The formulations can include a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, histidine, methionine, NaCl, and polysorbate. In some embodiments, the formulation can be at a pH between 5.3 and 6.3, which may or may not be accomplished by the addition of the histidine, methionine, NaCl, and/or polysorbate. In some embodiments, the antibody can be an anti-Gal3 antibody. In some embodiments, the antibody can be an anti-Gal3 antibody disclosed herein, or otherwise known in the art, such as those disclosed in WO 2020/160156. In some embodiments, the antibody comprises a heavy chain variable domain (VH) region having a sequence at least 80%, 85%, 90%, 95%, 99%, or 100% identical to that of SEQ ID NO: 8. In some embodiments, the antibody comprises a light chain variable domain (VL) region having a sequence at least 80%, 85%, 90%, 95%, 99%, or 100% identical to that of SEQ ID NO: 9. In some embodiments, the formulation can also include additional ingredients and/or excipients to those listed, or exclude one or more of the positively recited options. In some embodiments, the ingredients and/or excipients can be replaced or used additionally with one or more alternatives that function to achieve the same result. In some embodiments, the histidine can be replaced with an alternative buffer with an appropriate pKa. In some embodiments, the histidine can be replaced with an alternative that has the same buffer capacity. In some embodiments, the histidine can be replaced with another amino acid. In some embodiments, the histidine can be replaced with an alternative that exhibits the same or similar antibody protective effects. In some embodiments, the histidine can be replaced with an alternative that exhibits the same or similar capacity to reduce aggregation of the antibody. In some embodiments, the histidine can be replaced with an alternative that has the same or similar cryoprotective capabilities. In some embodiments, the methionine can be replaced with an alternative buffer with an appropriate pKa. In some embodiments, the methionine can be replaced with an alternative that has the same buffer capacity. In some embodiments, the methionine can be replaced with another amino acid. In some embodiments, the methionine can be replaced with an alternative that has the same or similar antioxidant effects. In some embodiments, the methionine can be replaced with an alternative that has the same antibody protective effects. In some embodiments, the methionine can be replaced by an alternative that has the same or similar protein stabilization effects. In some embodiments, the methionine can be replaced by an alternative that exhibits the same or similar capacity to reduce aggregation of the antibody, including alternatives that may exhibit any one or more of the properties provided herein. The alternatives for histidine and/or methionine may be any of those provided herein, such as arginine or glycine, or otherwise known in the art. In some embodiments, the NaCl can be replaced with another salt. In some embodiments, the NaCl can be replaced with an alternative that has the same or similar aqueous solubility. In some embodiments, the NaCl can be replaced with an alternative that has the same or similar effect on formulation isotonicity. In some embodiments, the NaCl can be replaced with an alternative that has the same or similar protein stabilization effects, including alternatives that may exhibit any one or more of the properties provided herein. The alternative for NaCl may be any of those provided herein, such as other chloride salts, other sodium salts, ascorbate salts, acetate salts, phosphate salts, citrate salts, Tris salts, or succinate salts, or otherwise known in the art. In some embodiments, the polysorbate can be replaced with another surfactant and/or detergent. In some embodiments, the polysorbate can be replaced with an alternative that has the same or similar surfactant ability/effect. In some embodiments, the polysorbate can be replaced with an alternative that has the same or similar capability for solubilizing antibodies and/or other excipients. In some embodiments, the polysorbate can be replaced with an alternative that has the same or similar capacity to reduce aggregation of the antibody. In some embodiments, the polysorbate can be replaced with an alternative that has the same or similar protein stabilization effects, including alternatives that may exhibit any one or more of the properties provided herein. The alternative for polysorbate may be any of those provided herein, such as poloxamer 188, or otherwise known in the art. In some embodiments, the pH can be acidic, basic, or neutral. In some embodiments, the pH can be basic. In some embodiments, the pH can be varied. In some embodiments, the pH can be increased or decreased in line with the ingredients, excipients, and/or buffers used in the formulation and the particulars of the antibody species used and/or the amount of antibody, ingredients, or excipients used. In some embodiments, the pH can be increased or decreased to a desired pH after adding the antibody, ingredients, or excipients. The alternatives contemplated herein may be any one or more of the excipients, diluents, salts, buffers, and the like, provided throughout the disclosure.

For any of the embodiments of the pharmaceutical antibody formulations provided herein, the histidine is L-histidine, D-histidine, or racemic histidine. For any of the embodiments of the pharmaceutical antibody formulations provided herein, the histidine is racemic histidine. For any of the embodiments of the pharmaceutical antibody formulations provided herein, the histidine is D-histidine. In some embodiments, the histidine can be replaced with an alternative buffer with an appropriate pKa. In some embodiments, the histidine can be replaced with an alternative that has the same buffer capacity. In some embodiments, the histidine can be replaced with another amino acid. In some embodiments, the histidine can be replaced with an alternative that exhibits the same or similar antibody protective effects. In some embodiments, the histidine can be replaced with an alternative that exhibits the same or similar capacity to reduce aggregation of the antibody. In some embodiments, the histidine can be replaced with an alternative that has the same or similar cryoprotective capabilities, including alternatives that may exhibit any one or more of the properties provided herein. The alternatives for histidine may be any of those provided herein, such as arginine or glycine, or otherwise known in the art.

For any of the embodiments of the pharmaceutical antibody formulations provided herein, the histidine may be present in a concentration that is, is about, is not more than, or is not less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mM, or any concentration within a range defined by any two of the aforementioned concentrations. In some embodiments, the histidine is present at 10 to 50 mM, e.g. 10, 15, 20, 25, 30, 35, 40, 45, or 50 mM. In some embodiments, the histidine is present at 20 mM or about 20 mM. In some embodiments, where the histidine is L-histidine, the L-histidine is present at 10 to 50 mM, e.g. 10, 15, 20, 25, 30, 35, 40, 45, or 50 mM. In some embodiments, where the histidine is L-histidine, the L-histidine is present at 20 mM or about 20 mM.

For any of the embodiments of the pharmaceutical antibody formulations provided herein, the methionine is L-methionine, D-methionine, or racemic methionine. For any of the embodiments of the pharmaceutical antibody formulations provided herein, the methionine is racemic methionine. For any of the embodiments of the pharmaceutical antibody formulations provided herein, the methionine is D-methionine. In some embodiments, the methionine can be replaced with an alternative buffer with an appropriate pKa. In some embodiments, the methionine can be replaced with an alternative that has the same buffer capacity. In some embodiments, the methionine can be replaced with another amino acid. In some embodiments, the methionine can be replaced with an alternative that has the same or similar antioxidant effects. In some embodiments, the methionine can be replaced with an alternative that has the same antibody protective effects. In some embodiments, the methionine can be replaced by an alternative that has the same or similar protein stabilization effects. In some embodiments, the methionine can be replaced by an alternative that exhibits the same or similar capacity to reduce aggregation of the antibody, including alternatives that may exhibit any one or more of the properties provided herein. The alternatives for methionine may be any of those provided herein, such as arginine or glycine, or otherwise known in the art.

For any of the embodiments of the pharmaceutical antibody formulations provided herein, the methionine may be present in a concentration that is, is about, is not more than, or is not less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mM, or any concentration within a range defined by any two of the aforementioned concentrations. In some embodiments, the methionine is present at 2 to 10 mM, e.g. 2, 3, 4, 5, 6, 7, 8, 9, or 10 mM. In some embodiments, the methionine is present at 5 mM or about 5 mM.

For any of the embodiments of the pharmaceutical antibody formulations provided herein, the NaCl may be present in a concentration that is, is about, is not more than, or is not less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mM, or any concentration within a range defined by any two of the aforementioned concentrations. In some embodiments, the NaCl is present at 50 to 150 mM, e.g. 50, 60, 70, 80, 90, 100, 110, 120, 130, 140, or 150 mM. In some embodiments, the NaCl is present at 100 mM. In some embodiments, the NaCl can be replaced with another salt. In some embodiments, the NaCl can be replaced with an alternative that has the same or similar aqueous solubility. In some embodiments, the NaCl can be replaced with an alternative that has the same or similar effect on formulation isotonicity. In some embodiments, the NaCl can be replaced with an alternative that has the same or similar protein stabilization effects, including alternatives that may exhibit any one or more of the properties provided herein. The alternative for NaCl may be any of those provided herein, such as other chloride salts, other sodium salts, ascorbate salts, acetate salts, phosphate salts, citrate salts, Tris salts, or succinate salts, or otherwise known in the art.

For any of the embodiments of the pharmaceutical antibody formulations provided herein, the polysorbate comprises polysorbate 20, polysorbate 40, polysorbate 60, polysorbate 80, or any combination thereof. In some embodiments, the polysorbate comprises, consists essentially of, or consists of polysorbate 80. In some embodiments, the polysorbate may be present in a concentration that is, is about, is not more than, or is not less than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any combination within a range within any two of the aforementioned concentrations. In some embodiments, the polysorbate is present at 0.01% to 0.04%, e.g. 0.01%, 0.02%, 0.03%, or 0.04%. In some embodiments, the polysorbate is present at about 0.01% to about 0.04%, e.g. about 0.01%, about 0.02%, about 0.03%, or about 0.04%. In some embodiments, the polysorbate is present at 0.02% or about 0.02%. In some embodiments, where the polysorbate is polysorbate 80, the polysorbate 80 is present at 0.01% to 0.04%, e.g. 0.01%, 0.02%, 0.03%, or 0.04%. In some embodiments, where the polysorbate is polysorbate 80, the polysorbate 80 is present at about 0.01% to about 0.04%, e.g. about 0.01%, about 0.02%, about 0.03%, or about 0.04%. In some embodiments, the polysorbate 80 is present at 0.02% or about 0.02%. In some embodiments, the polysorbate can be replaced with another surfactant and/or detergent. In some embodiments, the polysorbate can be replaced with an alternative that has the same or similar surfactant ability/effect. In some embodiments, the polysorbate can be replaced with an alternative that has the same or similar capability for solubilizing antibodies and/or other excipients. In some embodiments, the polysorbate can be replaced with an alternative that has the same or similar capacity to reduce aggregation of the antibody. In some embodiments, the polysorbate can be replaced with an alternative that has the same or similar protein stabilization effects, including alternatives that may exhibit any one or more of the properties provided herein. The alternative for polysorbate may be any of those provided herein, such as poloxamer 188, or otherwise known in the art.

For any of the embodiments of the pharmaceutical antibody formulations provided herein, the pH is, is about, is not more than, or not less than 5.3, 5.4, 5.5, 5.6, 5.7, 5.8, 5.9, 6.0, 6.1, 6.2, or 6.3. In some embodiments, the pH is about 5.8. In some embodiments, the pH is 5.8. In some embodiments, the pH can be acidic, basic, or neutral. In some embodiments, the pH can be varied. In some embodiments, the pH can be increased or decreased in line with the ingredients, excipients, and/or buffers used in the formulation and the particulars of the antibody species used and/or the amount of antibody, ingredients, or excipients used. In some embodiments, the pH can be increased or decreased to a desired pH after adding the antibody, ingredients, or excipients.

For any of the embodiments of the pharmaceutical antibody formulations provided herein, the formulations further comprise one or more sugars or one or more sugar alcohols, or both, such as the sugars or sugar alcohols disclosed herein or otherwise known in the art. In some embodiments, the one or more sugars comprises sucrose. In some embodiments, the one or more sugar alcohols comprise mannitol. In some embodiments, the formulations comprise sucrose or mannitol, or both. In some embodiments, the formulations comprise sucrose and mannitol. In some embodiments, the sucrose and/or mannitol can be replaced with another sugar and/or sugar alcohol. In some embodiments, the sucrose and/or mannitol can be replaced with an alternative that has the same or similar antibody protective effects. In some embodiments, the sucrose and/or mannitol can be replaced with an alternative that exhibits the same or similar capacity to reduce aggregation of the antibody. In some embodiments, the sucrose and/or mannitol can be replaced with an alternative that has the same or similar cryoprotective capabilities. In some embodiments, the sucrose and/or mannitol can be replaced with an alternative that have the same effect on isotonicity, including alternatives that may exhibit any one or more of the properties provided herein. The alternative for sucrose and/or mannitol may be any of those provided herein, such as sorbitol, trehalose, dextrose, dextran, or dextran 40, or otherwise known in the art.

In some embodiments, the one or more sugars or one or more sugar alcohols may be present in a concentration that is, is about, is not more than, or is not less than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any combination within a range within any two of the aforementioned concentrations. In some embodiments, the one or more sugars or one or more sugar alcohols are present at 2% to 5%, e.g. 2%, 3%, 4%, or 5%. In some embodiments, the one or more sugars or one or more sugar alcohols are present at about 2% to about 5%, e.g. about 2%, about 3%, about 4%, or about 5%. In some embodiments where the sugar is sucrose, the sucrose is present at 2% to 5% or about 2% to 5%. In some embodiments where the sugar alcohol is mannitol, the mannitol is present at 2% to 5% or about 2% to 5%.

For any of the embodiments of the pharmaceutical antibody formulations provided herein, the formulation is configured for parenteral administration. In some embodiments, the formulation is configured for subcutaneous administration. In embodiments where the formulation is configured for subcutaneous administration, the formulation may comprise one or more sugars and/or one or more sugar alcohols. In some embodiments, the formulation configured for subcutaneous administration comprises sucrose or mannitol, or both. In some embodiments, the formulation is configured for intravenous administration. In embodiments where the formulation is configured for intravenous administration, the formulation may not comprise one or more sugars and/or one or more sugar alcohols. In some embodiments, the formulation configured for intravenous administration does not comprise sucrose or mannitol, or both.

For any of the embodiments of the pharmaceutical antibody formulations provided herein, the antibody may be present at an amount that is, is about, is not more than, or is not less than 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3650, 3700, 3750, 3800, 4000, 4200, 4400, 4600, 4800, 5000, 5500, 6000, 6500, 7000, 7500, 8000, 8500, 9000, or 10000 mg as a unit dose, or any amount within a range defined by any two of the aforementioned amounts. In some embodiments, the antibody is present at an amount of 70 to 7500 mg as a unit dose. In some embodiments, the antibody is present at an amount of one of: 70 mg, 75 mg, 140 mg, 200 mg, 420 mg, 450 mg, 700 mg, 1500 mg, 2100 mg, 3750 mg, 5000 mg, or 7500 mg as a unit dose, or any amount within a range defined by any two of the aforementioned amounts. In some embodiments, the antibody is present at an amount of 70 mg. In some embodiments, the antibody is present at an amount of 75 mg. In some embodiments, the antibody is present at an amount of 140 mg. In some embodiments, the antibody is present at an amount of 200 mg. In some embodiments, the antibody is present at an amount of 420 mg. In some embodiments, the antibody is present at an amount of 450 mg. In some embodiments, the antibody is present at an amount of 700 mg. In some embodiments, the antibody is present at an amount of 1500 mg. In some embodiments, the antibody is present at an amount of 2100 mg. In some embodiments, the antibody is present at an amount of 3750 mg. In some embodiments, the antibody is present at an amount of 5000 mg. In some embodiments, the antibody is present at an amount of 7500 mg. In some embodiments, the antibody is present in the formulation at a concentration of one of: 1 mg/mL, 5 mg/mL, 10 mg/mL, 20 mg/mL, 40 mg/mL, or 50 mg/mL, or any concentration within a range defined by any two of the aforementioned concentrations. In some embodiments, the antibody is present at a concentration of 1 mg/mL. In some embodiments, the antibody is present at a concentration of 5 mg/mL. In some embodiments, the antibody is present at a concentration of 10 mg/mL. In some embodiments, the antibody is present at a concentration of 20 mg/mL. In some embodiments, the antibody is present at a concentration of 40 mg/mL. In some embodiments, the antibody is present at a concentration of 50 mg/mL.

In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is one of: 70 mg, 75 mg, 140 mg, 200 mg, 420 mg, 450 mg, 700 mg, 1500 mg, 2100 mg, 3750 mg, 5000 mg, or 7500 mg as a unit dose, or any amount within a range defined by any two of the aforementioned amounts. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 70 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 75 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 140 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 200 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 420 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 450 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 700 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 1500 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 2100 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 3750 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 5000 mg as a unit dose. In some embodiments of the pharmaceutical antibody formulations, L-histidine is present at about 20 mM, methionine is present at about 5 mM, NaCl is present at about 100 mM, polysorbate 80 is present at about 0.02%, sucrose is present at 2-5%, mannitol is present at 2-5%, the pH of the formulation is about 5.8, and the therapeutically effective amount of the antibody is 7500 mg as a unit dose.

In some embodiments, the pharmaceutical antibody formulation comprises a therapeutically effective amount of an antibody. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg, or any amount as a unit dose within a range defined by any two of the aforementioned amounts. In some embodiments, the antibody is present at an amount of 70 mg. In some embodiments, the antibody is present at an amount of 75 mg. In some embodiments, the antibody is present at an amount of 140 mg. In some embodiments, the antibody is present at an amount of 200 mg. In some embodiments, the antibody is present at an amount of 420 mg. In some embodiments, the antibody is present at an amount of 450 mg. In some embodiments, the antibody is present at an amount of 700 mg. In some embodiments, the antibody is present at an amount of 1500 mg. In some embodiments, the antibody is present at an amount of 2100 mg. In some embodiments, the antibody is present at an amount of 3750 mg. In some embodiments, the antibody is present at an amount of 5000 mg. In some embodiments, the antibody is present at an amount of 7500 mg. In some embodiments, the pharmaceutical antibody formulation further comprises L-histidine present at 20 mM, methionine present at 5 mM, NaCl present at 100 mM, polysorbate 80 present at 0.02%. In some embodiments, the pH of the formulation is about 5.8. In some embodiments, the pharmaceutical antibody formulation further comprises sucrose and/or mannitol. In some embodiments, sucrose is present in the formulation at 2-5%. In some embodiments, mannitol is present in the formulation at 2-5%.

In some embodiments of the pharmaceutical antibody formulations disclosed herein, the formulation is configured for parenteral administration. In some embodiments, the formulation is configured for subcutaneous administration. In some embodiments, the formulation configured for subcutaneous administration comprises sucrose or mannitol, or both. In some embodiments, the formulation is configured for intravenous administration. In some embodiments, the formulation configured for intravenous administration does not comprise sucrose or mannitol, or both.

As applied to any of the embodiments of the pharmaceutical antibody formulations disclosed herein, the pharmaceutical antibody formulation is prepared at a concentration of antibody that is, is about, is not more than, or is more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL, or any concentration within a range defined by any two of the aforementioned concentrations. In some embodiments, the pharmaceutical antibody formulation is prepared at a concentration of 0.3, 0.8, 2.8, 8.4, or 10 mg/mL, or about 0.3, about 0.8, about 2.8, about 8.4, or about 10 mg/mL. In some embodiments, the pharmaceutical antibody formulation is prepared at a concentration of 20 mg/mL or about 20 mg/mL. In some embodiments, the pharmaceutical antibody formulation is prepared at a concentration of 50 mg/mL or about 50 mg/mL.

As applied to any of the embodiments disclosed herein, the pharmaceutical antibody formulation remains at least 60%, 65%, 70%, 75%, 80%, 85%, 90%, 95%, 99%, or 100% stable over 3 months. In some embodiments, the pharmaceutical antibody formulation remains at least 60%, 65%, 70%, 75%, 80%, 85%, 90%, 95%, 99%, or 100% stable over 3 months at either 5° C. or 25° C./60% relative humidity (RH).

In some embodiments of the pharmaceutical antibody formulations disclosed herein, the antibody comprises a heavy chain variable domain (VH) region having a sequence at least 80%, 85%, 90%, 95%, 99%, or 100% identical to that of SEQ ID NO: 8. In some embodiments, the antibody comprises a light chain variable domain (VL) region having a sequence at least 80%, 85%, 90%, 95%, 99%, or 100% identical to that of SEQ ID NO: 9. In some embodiments, the antibody comprises a VH region having a sequence at least 80%, 85%, 90%, 95%, 99%, or 100% identical to that of SEQ ID NO: 8, and wherein the antibody comprises a VL region having a sequence at least 80%, 85%, 90%, 95%, 99%, or 100% identical to that of SEQ ID NO: 9. In some embodiments, the antibody comprises a VH region having a sequence of SEQ ID NO: 8. In some embodiments, the antibody comprises a VL region having a sequence of SEQ ID NO: 9. In some embodiments, the antibody comprises a VH region having a sequence of SEQ ID NO: 8, and wherein the antibody comprises a VL region having a sequence of SEQ ID NO: 9. In some embodiments, the antibody comprises a heavy chain (HC) having a sequence at least 80%, 85%, 90%, 95%, 99%, or 100% identical to that of SEQ ID NO: 10. In some embodiments, the antibody comprises a light chain (LC) having a sequence at least 80%, 85%, 90%, 95%, 99%, or 100% identical to that of SEQ ID NO: 11. In some embodiments, the antibody comprises an HC having a sequence of SEQ ID NO: 10. In some embodiments, the antibody comprises an LC having a sequence of SEQ ID NO: 11. In some embodiments, exemplary VH, VL, HC, and LC polypeptide sequences are depicted in FIG. 2B. In some embodiments, the antibody is TB006 (4A11.H3L1, IMT006a, IMT006-5). The % identity of two sequences is well understood in the art and can be calculated by the number of conserved amino acids or nucleotides relative to the length of the sequences.

In some embodiments of the pharmaceutical antibody formulations disclosed herein, the antibody or a component thereof is encoded by one or more nucleic acids. In some embodiments, the antibody comprises a VH that is encoded by a nucleic acid sequence having at least 80%, 85%, 90%, 95%, 99%, or 100% identical to that of SEQ ID NO: 12. In some embodiments, the antibody comprises a VL that is encoded by a nucleic acid sequence having at least 80%, 85%, 90%, 95%, 99%, or 100% identical to that of SEQ ID NO: 13. In some embodiments, the antibody comprises a VH that is encoded by a nucleic acid sequence of SEQ ID NO: 12. In some embodiments, the antibody comprises a VL that is encoded by a nucleic acid sequence of SEQ ID NO: 13. In some embodiments, the antibody comprises an HC that is encoded by a nucleic acid sequence having at least 80%, 85%, 90%, 95%, 99%, or 100% identical to that of SEQ ID NO: 14. In some embodiments, the antibody comprises an LC that is encoded by a nucleic acid sequence having at least 80%, 85%, 90%, 95%, 99%, or 100% identical to that of SEQ ID NO: 15. In some embodiments, the antibody comprises an HC that is encoded by a nucleic acid sequence of SEQ ID NO: 14. In some embodiments, the antibody comprises an LC that is encoded by a nucleic acid sequence of SEQ ID NO: 15. In some embodiments, exemplary VH, VL, HC, and LC nucleic acid sequences are depicted in FIG. 2C. The % identity of two sequences is well understood in the art and can be calculated by the number of conserved amino acids or nucleotides relative to the length of the sequences.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multi-particulate formulations (e.g., nanoparticle formulations), and mixed immediate and controlled release formulations.

In some embodiments, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-(hydroxymethyl)aminomethane; and buffers such as citrate/dextrose, sodium bicarbonate and ammonium chloride. Such acids, bases and buffers are included in an amount required to maintain pH of the formulation in an acceptable range.

In some embodiments, the pharmaceutical formulations include one or more salts in an amount required to bring osmolality of the formulation into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the pharmaceutical formulations further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the formulation to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some embodiments, the pharmaceutical formulation is formulated for administration to a subject by one or more administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some embodiments, the pharmaceutical formulation described herein is formulated for parenteral (e.g., intravenous, subcutaneous, intramuscular, intraarterial, intradermal, intraperitoneal, intravitreal, intracerebral, or intracerebroventricular) administration. In some embodiments, the pharmaceutical antibody formulation is formulated for intravenous administration. In some embodiments, the pharmaceutical antibody formulation is formulated for subcutaneous administration. Proper formulation is dependent upon the route of administration chosen. Techniques for formulation and administration of the compounds described herein are known to those skilled in the art.

In some embodiments, the pharmaceutical antibody formulation comprises a therapeutically effective amount of an antibody.

In some embodiments, the pharmaceutical antibody formulation comprises a therapeutically effective amount of an antibody, histidine, methionine, NaCl, and polysorbate.

In some embodiments, the pharmaceutical antibody formulation comprises a therapeutically effective amount of an antibody, histidine, methionine, NaCl, and polysorbate, where the formulation is at a pH between 5.3 and 6.3.

In some embodiments, the pharmaceutical antibody formulation comprises a therapeutically effective amount of an antibody, histidine, methionine, NaCl, and polysorbate, where the formulation is at a pH between 5.3 and 6.3. In some embodiments, the histidine is L-histidine. In some embodiments, the polysorbate is polysorbate 80. In some embodiments, the histidine is L-histidine and the polysorbate is polysorbate 80. In some embodiments, the pharmaceutical antibody formulation comprises a therapeutically effective amount of an antibody, histidine, methionine, NaCl, and polysorbate, where the formulation is at a pH between 5.3 and 6.3, where the histidine is L-histidine. In some embodiments, the pharmaceutical antibody formulation comprises a therapeutically effective amount of an antibody, histidine, methionine, NaCl, and polysorbate, where the formulation is at a pH between 5.3 and 6.3, where the polysorbate is polysorbate 80. In some embodiments, the pharmaceutical antibody formulation comprises a therapeutically effective amount of an antibody, histidine, methionine, NaCl, and polysorbate, where the formulation is at a pH between 5.3 and 6.3, where the histidine is L-histidine and the polysorbate is polysorbate 80.

In some embodiments, the pharmaceutical antibody formulation comprises a therapeutically effective amount of an antibody, histidine, methionine, NaCl, and polysorbate, where the formulation is at a pH between 5.3 and 6.3, where the histidine is present at 10 to 50 mM. In some embodiments, the pharmaceutical antibody formulation comprises a therapeutically effective amount of an antibody, histidine, methionine, NaCl, and polysorbate, where the formulation is at a pH between 5.3 and 6.3, where the histidine is present at 20 mM. In some embodiments, the pharmaceutical antibody formulation comprises a therapeutically effective amount of an antibody, histidine, methionine, NaCl, and polysorbate, where the formulation is at a pH between 5.3 and 6.3, where the methionine is present at 2 to 10 mM. In some embodiments, the pharmaceutical antibody formulation comprises a therapeutically effective amount of an antibody, histidine, methionine, NaCl, and polysorbate, where the formulation is at a pH between 5.3 and 6.3, where the methionine is present at 5 mM. In some embodiments, the pharmaceutical antibody formulation comprises a therapeutically effective amount of an antibody, histidine, methionine, NaCl, and polysorbate, where the formulation is at a pH between 5.3 and 6.3, where the NaCl is present at 50 to 150 mM. In some embodiments, the pharmaceutical antibody formulation comprises a therapeutically effective amount of an antibody, histidine, methionine, NaCl, and polysorbate, where the formulation is at a pH between 5.3 and 6.3, where the NaCl is present at 100 mM. In some embodiments, the pharmaceutical antibody formulation comprises a therapeutically effective amount of an antibody, histidine, methionine, NaCl, and polysorbate, where the formulation is at a pH between 5.3 and 6.3, where the polysorbate is present at 0.01 to 0.04%. In some embodiments, the pharmaceutical antibody formulation comprises a therapeutically effective amount of an antibody, histidine, methionine, NaCl, and polysorbate, where the formulation is at a pH between 5.3 and 6.3, where the polysorbate is present at 0.02%. In some embodiments, the histidine is L-histidine. In some embodiments, the polysorbate is polysorbate 20, polysorbate 40, polysorbate 60, or polysorbate 80. In some embodiments, the polysorbate is polysorbate 80. In some embodiments, the pH is 5.8. In some embodiments, the pharmaceutical antibody formulation further comprises sucrose. In some embodiments, the sucrose is present at 2% to 5%. In some embodiments, the pharmaceutical antibody formulation further comprises mannitol. In some embodiments, the mannitol is present at 2% to 5%.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, histidine, methionine, NaCl, and polysorbate, where the formulation is at a pH between 5.3 and 6.3.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, histidine present at 20 mM, methionine present at 5 mM, NaCl present at 100 mM, and polysorbate present at 0.02%, where the formulation is at a pH between 5.3 and 6.3.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, histidine present at 20 mM, methionine present at 5 mM, NaCl present at 100 mM, and polysorbate present at 0.02%, where the formulation is at a pH of about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, L-histidine present at 20 mM, methionine present at 5 mM, NaCl present at 100 mM, and polysorbate 80 present at 0.02%, where the formulation is at a pH between 5.3 and 6.3.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, L-histidine present at 20 mM, methionine present at 5 mM, NaCl present at 100 mM, and polysorbate 80 present at 0.02%, where the formulation is at a pH of about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg, histidine present at 20 mM, methionine present at 5 mM, NaCl present at 100 mM, and polysorbate present at 0.02%, where the formulation is at a pH between 5.3 and 6.3.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg histidine present at 20 mM, methionine present at 5 mM, NaCl present at 100 mM, and polysorbate present at 0.02%, where the formulation is at a pH of about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg L-histidine present at 20 mM, methionine present at 5 mM, NaCl present at 100 mM, and polysorbate 80 present at 0.02%, where the formulation is at a pH between 5.3 and 6.3.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg L-histidine present at 20 mM, methionine present at 5 mM, NaCl present at 100 mM, and polysorbate 80 present at 0.02%, where the formulation is at a pH of about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg, histidine present at 20 mM, methionine present at 5 mM, NaCl present at 100 mM, and polysorbate present at 0.02%, where the formulation is at a pH between 5.3 and 6.3.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg histidine present at 20 mM, methionine present at 5 mM, NaCl present at 100 mM, and polysorbate present at 0.02%, where the formulation is at a pH of about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg L-histidine present at 20 mM, methionine present at 5 mM, NaCl present at 100 mM, and polysorbate 80 present at 0.02%, where the formulation is at a pH between 5.3 and 6.3.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount as a unit dose of 70 mg, 75 mg, 140 mg, 200 mg, 420 mg, 450 mg, 700 mg, 1500 mg, 2100 mg, 3750 mg, 5000 mg, or 7500 mg L-histidine present at 20 mM, methionine present at 5 mM, NaCl present at 100 mM, and polysorbate 80 present at 0.02%, where the formulation is at a pH of about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 70 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 75 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 140 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 200 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 420 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 450 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 700 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 1500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 2100 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 3750 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 5000 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 7500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 70 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 75 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 140 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 200 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 420 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 450 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 700 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 1500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 2100 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 3750 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 5000 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 7500 mg as a unit dose, L-histidine present at 20 mM, methionine present at 5 mM, NaCl present at 100 mM, polysorbate 80 present at 0.02% and where the pH is about 5.8.

### Exemplary Articles of Manufacture and Kits

In some embodiments, sterile vials comprising pharmaceutical antibody formulations are provided, where the formulations can include a therapeutically effective amount of an antibody. In some embodiments, the sterile vials comprise any one of the pharmaceutical antibody formulations disclosed herein. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2) having the sequence of SEQ ID NO: 6; and an LCDR3 having the sequence of SEQ ID NO: 7. In some embodiments, the pharmaceutical antibody formulations can further comprise histidine, methionine, NaCl, and polysorbate. In some embodiments, the formulation can be at a pH between 5.3 and 6.3. In some embodiments, the antibody can be an anti-Gal3 antibody. In some embodiments, the antibody can be an anti-Gal3 antibody disclosed herein, or otherwise known in the art, such as those disclosed in WO 2020/160156. In some embodiments, the antibody comprises a heavy chain variable domain (VH) region having a sequence at least 80%, 85%, 90%, 95%, 99%, or 100% identical to that of SEQ ID NO: 8. In some embodiments, the antibody comprises a light chain variable domain (VL) region having a sequence at least 80%, 85%, 90%, 95%, 99%, or 100% identical to that of SEQ ID NO: 9. In some embodiments, the formulation can also include additional ingredients and/or excipients to those listed, or exclude one or more of the positively recited options. In some embodiments, the ingredients and/or excipients can be replaced or used additionally with one or more alternatives that function to achieve the same result. In some embodiments, the histidine can be replaced with an alternative buffer with an appropriate pKa. In some embodiments, the histidine can be replaced with an alternative that has the same buffer capacity. In some embodiments, the histidine can be replaced with another amino acid. In some embodiments, the histidine can be replaced with an alternative that exhibits the same or similar antibody protective effects. In some embodiments, the histidine can be replaced with an alternative that exhibits the same or similar capacity to reduce aggregation of the antibody. In some embodiments, the histidine can be replaced with an alternative that has the same or similar cryoprotective capabilities, including alternatives that may exhibit any one or more of the properties provided herein. In some embodiments, the methionine can be replaced with an alternative buffer with an appropriate pKa. In some embodiments, the methionine can be replaced with an alternative that has the same buffer capacity. In some embodiments, the methionine can be replaced with another amino acid. In some embodiments, the methionine can be replaced with an alternative that has the same or similar antioxidant effects. In some embodiments, the methionine can be replaced with an alternative that has the same antibody protective effects. In some embodiments, the methionine can be replaced by an alternative that has the same or similar protein stabilization effects. In some embodiments, the methionine can be replaced by an alternative that exhibits the same or similar capacity to reduce aggregation of the antibody, including alternatives that may exhibit any one or more of the properties provided herein. The alternatives for histidine and/or methionine may be any of those provided herein, such as arginine or glycine, or otherwise known in the art. In some embodiments, the NaCl can be replaced with another salt. In some embodiments, the NaCl can be replaced with an alternative that has the same or similar aqueous solubility. In some embodiments, the NaCl can be replaced with an alternative that has the same or similar effect on formulation isotonicity. In some embodiments, the NaCl can be replaced with an alternative that has the same or similar protein stabilization effects, including alternatives that may exhibit one or more of the properties provided herein. The alternative for NaCl may be any of those provided herein, such as other chloride salts, other sodium salts, ascorbate salts, acetate salts, phosphate salts, citrate salts, Tris salts, or succinate salts, or otherwise known in the art. In some embodiments, the polysorbate can be replaced with another surfactant and/or detergent. In some embodiments, the polysorbate can be replaced with an alternative that has the same or similar surfactant ability/effect. In some embodiments, the polysorbate can be replaced with an alternative that has the same or similar capability for solubilizing antibodies and/or other excipients. In some embodiments, the polysorbate can be replaced with an alternative that has the same or similar capacity to reduce aggregation of the antibody. In some embodiments, the polysorbate can be replaced with an alternative that has the same or similar protein stabilization effects, including alternatives that may exhibit one or more of the properties provided herein. The alternative for polysorbate may be any of those provided herein, such as poloxamer 188, or otherwise known in the art. In some embodiments, the pH can be acidic. In some embodiments, the pH can be basic. In some embodiments, the pH can be varied. In some embodiments, the pH can be increased or decreased in line with the ingredients, excipients, and/or buffers used in the formulation and the particulars of the antibody species used and/or the amount of antibody, ingredients, or excipients used. In some embodiments, the pH can be increased or decreased to a desired pH after adding the antibody, ingredients, or excipients. The alternatives contemplated herein may be any one or more of the excipients, diluents, salts, buffers, and the like, provided throughout the disclosure.

For any of the embodiments of the sterile vials comprising pharmaceutical antibody formulations provided herein, the histidine is L-histidine, D-histidine, or racemic histidine. For any of the embodiments of the pharmaceutical antibody formulations provided herein, the histidine is racemic histidine. For any of the embodiments of the pharmaceutical antibody formulations provided herein, the histidine is D-histidine. In some embodiments, the histidine can be replaced with an alternative buffer with an appropriate pKa. In some embodiments, the histidine can be replaced with an alternative that has the same buffer capacity. In some embodiments, the histidine can be replaced with another amino acid. In some embodiments, the histidine can be replaced with an alternative that exhibits the same or similar antibody protective effects. In some embodiments, the histidine can be replaced with an alternative that exhibits the same or similar capacity to reduce aggregation of the antibody. In some embodiments, the histidine can be replaced with an alternative that has the same or similar cryoprotective capabilities, including alternatives that may exhibit one or more of the properties provided herein. The alternatives for histidine may be any of those provided herein, such as arginine or glycine, or otherwise known in the art.

For any of the embodiments of the sterile vials comprising pharmaceutical antibody formulations provided herein, the histidine is present at 10 to 50 mM, e.g. 10, 15, 20, 25, 30, 35, 40, 45, or 50 mM. In some embodiments, the histidine is present at 20 mM or about 20 mM. In some embodiments, where the histidine is L-histidine, the L-histidine is present at 10 to 50 mM, e.g. 10, 15, 20, 25, 30, 35, 40, 45, or 50 mM. In some embodiments, where the histidine is L-histidine, the L-histidine is present at 20 mM or about 20 mM.

For any of the embodiments of the sterile vials comprising pharmaceutical antibody formulations provided herein, the methionine is L-methionine. For any of the embodiments of the pharmaceutical antibody formulations provided herein, the methionine is racemic methionine. For any of the embodiments of the pharmaceutical antibody formulations provided herein, the methionine is D-methionine. In some embodiments, the methionine can be replaced with an alternative buffer with an appropriate pKa. In some embodiments, the methionine can be replaced with an alternative that has the same buffer capacity. In some embodiments, the methionine can be replaced with another amino acid. In some embodiments, the methionine can be replaced with an alternative that has the same or similar antioxidant effects. In some embodiments, the methionine can be replaced with an alternative that has the same antibody protective effects. In some embodiments, the methionine can be replaced by an alternative that has the same or similar protein stabilization effects. In some embodiments, the methionine can be replaced by an alternative that exhibits the same or similar capacity to reduce aggregation of the antibody, including alternatives that may exhibit one or more of the properties provided herein. The alternatives for methionine may be any of those provided herein, such as arginine or glycine, or otherwise known in the art.

For any of the embodiments of the sterile vials comprising pharmaceutical antibody formulations provided herein, the methionine is present at 2 to 10 mM, e.g. 2, 3, 4, 5, 6, 7, 8, 9, or 10 mM. In some embodiments, the methionine is present at 5 mM or about 5 mM.

For any of the embodiments of the sterile vials comprising pharmaceutical antibody formulations provided herein, the NaCl is present at 50 to 150 mM, e.g. 50, 60, 70, 80, 90, 100, 110, 120, 130, 140, or 150 mM. In some embodiments, the NaCl is present at 100 mM. In some embodiments, the NaCl can be replaced with another salt. In some embodiments, the NaCl can be replaced with an alternative that has the same or similar aqueous solubility. In some embodiments, the NaCl can be replaced with an alternative that has the same or similar effect on formulation isotonicity. In some embodiments, the NaCl can be replaced with an alternative that has the same or similar protein stabilization effects, including alternatives that may exhibit one or more of the properties provided herein. The alternative for NaCl may be any of those provided herein, such as other chloride salts, other sodium salts, ascorbate salts, acetate salts, phosphate salts, citrate salts, Tris salts, or succinate salts, or otherwise known in the art.

For any of the embodiments of the sterile vials comprising pharmaceutical antibody formulations provided herein, the polysorbate comprises polysorbate 20, polysorbate 40, polysorbate 60, polysorbate 80, or any combination thereof. In some embodiments, the polysorbate comprises, consists essentially of, or consists of polysorbate 80. In some embodiments, the polysorbate is present at 0.01% to 0.04%, e.g. 0.01%, 0.02%, 0.03%, or 0.04%. In some embodiments, the polysorbate is present at about 0.01% to about 0.04%, e.g. about 0.01%, about 0.02%, about 0.03%, or about 0.04%. In some embodiments, the polysorbate is present at 0.02% or about 0.02%. In some embodiments, where the polysorbate is polysorbate 80, the polysorbate 80 is present at 0.01% to 0.04%, e.g. 0.01%, 0.02%, 0.03%, or 0.04%. In some embodiments, where the polysorbate is polysorbate 80, the polysorbate 80 is present at about 0.01% to about 0.04%, e.g. about 0.01%, about 0.02%, about 0.03%, or about 0.04%. In some embodiments, the polysorbate 80 is present at 0.02% or about 0.02%. In some embodiments, the polysorbate can be replaced with another surfactant and/or detergent. In some embodiments, the polysorbate can be replaced with an alternative that has the same or similar surfactant ability/effect. In some embodiments, the polysorbate can be replaced with an alternative that has the same or similar capability for solubilizing antibodies and/or other excipients. In some embodiments, the polysorbate can be replaced with an alternative that has the same or similar capacity to reduce aggregation of the antibody. In some embodiments, the polysorbate can be replaced with an alternative that has the same or similar protein stabilization effects, including alternatives that may exhibit one or more of the properties provided herein. The alternative for polysorbate may be any of those provided herein, such as poloxamer 188, or otherwise known in the art.

For any of the embodiments of the sterile vials comprising pharmaceutical antibody formulations provided herein, the pH is about 5.8. In some embodiments, the pH is 5.8. In some embodiments, the pH can be acidic. In some embodiments, the pH can be basic. In some embodiments, the pH can be varied. In some embodiments, the pH can be increased or decreased in line with the ingredients, excipients, and/or buffers used in the formulation and the particulars of the antibody species used and/or the amount of antibody, ingredients, or excipients used. In some embodiments, the pH can be increased or decreased to a desired pH after adding the antibody, ingredients, or excipients.

For any of the embodiments of the sterile vials comprising pharmaceutical antibody formulations provided herein, the formulations further comprise one or more sugars or one or more sugar alcohols, or both, including any one of the sugars or sugar alcohols disclosed herein or otherwise known in the art. In some embodiments, the one or more sugars comprises sucrose. In some embodiments, the one or more sugar alcohols comprise mannitol. In some embodiments, the formulations comprise sucrose or mannitol, or both. In some embodiments, the formulations comprise sucrose and mannitol. In some embodiments, the sucrose and/or mannitol can be replaced with another sugar and/or sugar alcohol. In some embodiments, the sucrose and/or mannitol can be replaced with an alternative that has the same or similar antibody protective effects. In some embodiments, the sucrose and/or mannitol can be replaced with an alternative that exhibits the same or similar capacity to reduce aggregation of the antibody. In some embodiments, the sucrose and/or mannitol can be replaced with an alternative that has the same or similar cryoprotective capabilities. In some embodiments, the sucrose and/or mannitol can be replaced with an alternative that have the same effect on isotonicity, including alternatives that may exhibit one or more of the properties provided herein. The alternative for sucrose and/or mannitol may be any of those provided herein, such as sorbitol, trehalose, dextrose, dextran, or dextran 40, or otherwise known in the art.

In some embodiments, the one or more sugars or one or more sugar alcohols are present at 2% to 5%, e.g. 2%, 3%, 4%, or 5%. In some embodiments, the one or more sugars or one or more sugar alcohols are present at about 2% to about 5%, e.g. about 2%, about 3%, about 4%, or about 5%. In some embodiments where the sugar is sucrose, the sucrose is present at 2% to 5% or about 2% to 5%. In some embodiments where the sugar alcohol is mannitol, the mannitol is present at 2% to 5% or about 2% to 5%.

For any of the embodiments of the sterile vials comprising pharmaceutical antibody formulations provided herein, the formulation is configured for parenteral administration. In some embodiments, the formulation is configured for subcutaneous administration. In some embodiments, the formulation configured for subcutaneous administration comprises one or more sugars and/or one or more sugar alcohols. In some embodiments, the formulation configured for subcutaneous administration comprises sucrose or mannitol, or both. In some embodiments, the formulation is configured for intravenous administration. In some embodiments, the formulation configured for intravenous administration does not comprise one or more sugars and/or one or more sugar alcohols. In some embodiments, the formulation configured for intravenous administration does not comprise sucrose or mannitol, or both.

In some embodiments are sterile vials comprising a pharmaceutical antibody formulation comprising a therapeutically effective amount of an antibody, such as an anti-Gal3 antibody. In some embodiments, the sterile vials comprise any of the pharmaceutical antibody formulations disclosed herein. In some embodiments, the pharmaceutical antibody formulation comprises a therapeutically effective amount of an antibody. In some embodiments, the antibody is an anti-Gal3 antibody. In some embodiments, the antibody is any one of the anti-Gal3 antibodies disclosed herein or otherwise known in the art, such as those described in WO 2020/160156. In some embodiments, the antibody comprises an HCDR1 having the sequence of SEQ ID NO; 2, an HCDR2 having the sequence of SEQ ID NO: 3, an HCDR3 having the sequence of SEQ ID NO; 4, an LCDR1 having the sequence of SEQ ID NO: 5, an LCDR2 having the sequence of SEQ ID NO: 6; and an LCDR3 having the sequence of SEQ ID NO: 7. In some embodiments, the pharmaceutical antibody formulation in the sterile vial further comprises histidine, methionine, NaCl, and polysorbate. In some embodiments, the pharmaceutical antibody formulation in the sterile vial is at a pH between 5.3 and 6.3. In some embodiments, the sterile vial is a 1, 2, 3, 4, 5, 6, 7, 8, 9, or 10 mL sterile vial. In some embodiments, the sterile vial is a 5 mL sterile vial. In some embodiments, the sterile vial is a 10 mL sterile vial. In some embodiments, the sterile vial contains 1, 2, 3, 4, 5, 6, 7, 8, 9, or 10 mL of the pharmaceutical antibody formulation. In some embodiments, the sterile vial contains 2 mL or at least 2 mL of the pharmaceutical antibody formulation. In some embodiments, the sterile vial contains 8 mL or at least 8 mL of the pharmaceutical antibody formulation. In some embodiments, the pharmaceutical antibody formulation in the sterile vial is a concentrated form of any one of the pharmaceutical antibody formulations disclosed herein. In some embodiments, the concentrated form of the pharmaceutical antibody formulation in the sterile vial is at a concentration of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL. In some embodiments, the concentrated form of the pharmaceutical antibody formation in the sterile vial is at a concentration of 20 mg/mL or about 20 mg/mL or at least 20 mg/mL. In some embodiments, the concentrated form of the pharmaceutical antibody formulation in the sterile vial is at a concentration of 50 mg/mL or about 50 mg/mL or at least 50 mg/mL. In some embodiments, the sterile vial contains 1, 2, 3, 4, 5, 6, 7, 8, 9, or 10 mL of the concentrated form of the pharmaceutical antibody formulation, where the concentrated form of the pharmaceutical antibody formulation is at a concentration of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL of antibody. In some embodiments, the sterile vial comprises 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg of the antibody, or any amount of antibody within a range defined by any two of the aforementioned amounts. In some embodiments, the concentrated form of the pharmaceutical antibody formulation in the sterile vial is intended to be diluted 1×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, or 100× fold, or any fold within a range defined by any two of the aforementioned fold. In some embodiments, the concentrated form of the pharmaceutical antibody formulation in the sterile vial is intended to be diluted to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations. In some embodiments, the concentrated form of the pharmaceutical antibody formulation is intended to be diluted into a final volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes. In some embodiments, the concentrated form of the pharmaceutical antibody formulation is intended to be diluted into a final volume of 250 mL or 500 mL. In some embodiments, the concentrated form of the pharmaceutical antibody formulation in the sterile vial is intended to be diluted with saline. In some embodiments, the saline is 0.9% saline. In some embodiments, the pharmaceutical antibody formulation in the sterile vial is configured for parenteral administration. In some embodiments, the pharmaceutical antibody formulation in the sterile vial is configured for subcutaneous administration. In some embodiments, the pharmaceutical antibody formulation in the sterile vial configured for subcutaneous administration comprises sucrose or mannitol, or both. In some embodiments, the pharmaceutical antibody formulation in the sterile vial is configured for intravenous administration. In some embodiments, the pharmaceutical antibody formulation in the sterile vial configured for intravenous administration does not comprise sucrose or mannitol, or both. In some embodiments, the pharmaceutical antibody formulation remains 60%, 65%, 70%, 75%, 80%, 85%, 90%, 95%, 99%, or 100% stable over 3 months. In some embodiments, the pharmaceutical antibody formulation remains 60%, 65%, 70%, 75%, 80%, 85%, 90%, 95%, 99%, or 100% stable over 3 months at either 5° C. or 25° C./60% relative humidity (RH).

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 70 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 75 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 140 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 200 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 420 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 450 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 700 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 1500 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 2100 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 3750 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 5000 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 7500 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 70 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 75 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 140 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 200 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 420 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 450 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 700 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 1500 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 2100 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 3750 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 5000 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial comprises a pharmaceutical antibody formulation comprising a therapeutically effective amount of an antibody or a concentrated form of the pharmaceutical antibody formulation, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 7500 mg as a unit dose, L-histidine present at 20 mM, methionine present at 5 mM, NaCl present at 100 mM, polysorbate 80 present at 0.02% and where the pH is about 5.8. In some embodiments, the pharmaceutical antibody formulation is a concentrated form. In some embodiments, the pharmaceutical antibody formulation is at a concentration of 20 mg/mL or about 20 mg/mL of antibody. In some embodiments, the pharmaceutical antibody formulation is at a concentration of 50 mg/mL or about 50 mg/mL of antibody.

In some embodiments, the sterile vial can be substituted with a suitable alternative container, such as a tube, bag, pack, syringe, or dispenser. In some embodiments, the vial or alternative container may be contained within a kit for use. In some embodiments, the kit may contain identifying description, label, or instructions relating to its use in the methods disclosed herein. In some embodiments, the kit also includes a notice prescribed by a government agency regulating the manufacture, use, or sale of pharmaceuticals, denoting approval of the form of the drug for human or veterinary administration.

Any one of the disclosed formulations (including those stored in embodiments of sterile vials provided herein and those described in Examples 2-3) can include an anti-Gal3 antibody and have a pH of between 5.7-5.9 or about 5.7-5.9. In some embodiments, the formulation exhibits a pH of between 5.7-5.9 or about 5.7-5.9 when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a monomeric purity of 97.5-99.7% or about 97.5-99.7% as determined by size exclusion chromatography (SEC). In some embodiments, the formulation exhibits a monomeric purity of 97.5-99.7% or about 97.5-99.7% as determined by SEC when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a pI of 7.0 or about 7.0 as determined by capillary isoelectric focusing (cIEF). In some embodiments, the formulation exhibits a pI of 7.0 or about 7.0 as determined by capillary isoelectric focusing (cIEF) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a cIEF acidic peak of 15.0-54.7% or about 15.0-54.7%. In some embodiments, the formulation exhibits a cIEF acidic peak of 15.0-54.7% or about 15.0-54.7% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a cIEF main peak of 39.7-78.8% or about 39.7-78.8%. In some embodiments, the formulation exhibits a cIEF main peak of 39.7-78.8% or about 39.7-78.8% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a cIEF basic peak of 5.5-8.9% or about 5.5-8.9%. In some embodiments, the formulation exhibits a cIEF basic peak of 5.5-8.9% or about 5.5-8.9% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and have a peak of non-reduced (NR) monomer of 98.1-100% or about 98.1-100%. In some embodiments, the formulation exhibits a peak of non-reduced (NR) monomer of 98.1-100% or about 98.1-100% as determined by capillary electrophoresis (CE) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a peak of reduced (R) heavy chain and light chain (HC+LC) of 97.8-100% or about 97.8-100% as determined by CE. In some embodiments, the formulation exhibits a peak of reduced (R) heavy chain and light chain (HC+LC) of 97.8-100% or about 97.8-100% as determined by CE when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a dissociation constant (KD) of 1.7-4.2 or about 1.7-4.2 as determined by biolayer interferometry (BLI). In some embodiments, the formulation exhibits a dissociation constant (KD) of 1.7-4.2 or about 1.7-4.2 as determined by biolayer interferometry (BLI) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a pI of 7.0 or about 7.0 as determined by cIEF. In some embodiments, the formulation exhibits a pI of 7.0 or about 7.0 as determined by cIEF when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a cIEF acidic peak of 17-26% or about 17-26%. In some embodiments, the formulation exhibits a cIEF acidic peak of 17-26% or about 17-26% when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a cIEF main peak of 66-77% or about 66-7%. In some embodiments, the formulation exhibits a cIEF main peak of 66-77% or about 66-7% when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a cIEF basic peak of 6-8% or about 6-8%. In some embodiments, the formulation exhibits a cIEF basic peak of 6-8% or about 6-8% when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a monomeric purity of 99.3-99.5% or about 99.3-99.5% as determined by ultra-high performance liquid chromatography-size exclusion chromatography (UPLC-SEC). In some embodiments, the formulation exhibits a monomeric purity of 99.3-99.5% or about 99.3-99.5% as determined by ultra-high performance liquid chromatography-size exclusion chromatography (UPLC-SEC) when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a peak of non-reduced (NR) monomer of 98-99% or about 98-99% as determined by CE. In some embodiments, the formulation exhibits a peak of non-reduced (NR) monomer of 98-99% or about 98-99% as determined by CE when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a peak of reduced (R) heavy chain and light chain (HC+LC) of 99.1-100% or about 99.1-100% as determined by CE. In some embodiments, the formulation exhibits a peak of reduced (R) heavy chain and light chain (HC+LC) of 99.1-100% or about 99.1-100% as determined by CE when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a dissociation constant (KD) of 2.0-3.7 nM or about 2.0-3.7 nM as determined by biolayer interferometry (BLI). In some embodiments, the formulation exhibits a dissociation constant (KD) of 2.0-3.7 nM or about 2.0-3.7 nM as determined by biolayer interferometry (BLI) when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has an IC50 of 1.2-2.5 μg/mL or about 1.2-2.5 μg/mL as determined by ELISA. In some embodiments, the formulation exhibits an IC50 of 1.2-2.5 μg/mL or about 1.2-2.5 μg/mL as determined by ELISA when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has a pH of 5.8-5.9 or about 5.8-5.9. In some embodiments, the formulation exhibits a pH of 5.8-5.9 or about 5.8-5.9 when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and comprises an appearance of a clear, colorless solution essentially free of particles. In some embodiments, the formulation comprises an appearance of a clear, colorless solution essentially free of particles when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and has an osmolality of 226-231 mOsm/kg or about 226-231 mOsm/kg. In some embodiments, the formulation exhibits an osmolality of 226-231 mOsm/kg or about 226-231 mOsm/kg when stored at a) 5° C. for 1, 3, 6, 9, 12, or 18 months, or b) 25° C. for 1, 3, or 6 months. This can be any of the disclosed formulations or in other embodiments, any formulation that meets these properties.

Any one of the disclosed formulations (including those stored in embodiments of sterile vials provided herein and those described in Examples 2-3) can include an anti-Gal3 antibody and remains sterile and/or free of bacterial endotoxins. In some embodiments, the formulation remains sterile and/or free of bacterial endotoxins when stored at a) 5° C. for 1, 3, 6, 9, 12, or 18 months, or b) 25° C. for 1, 3, or 6 months. This can be any of the disclosed formulations or in other embodiments, any formulation that meets these properties.

In some embodiments, any of the pharmaceutical antibody formulations disclosed herein are administered for therapeutic applications. In some embodiments, these can be used for the treatment of a neurological disorder or proteopathy, such as Alzheimer's disease, or inflammation associated with said diseases.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 70 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 75 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 140 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 200 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 420 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 450 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 700 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 1500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 2100 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 3750 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 5000 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, where the antibody is present at an amount of 7500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 70 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 75 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 140 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 200 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 420 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 450 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 700 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 1500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 2100 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 3750 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 5000 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation administered for therapeutic applications comprises a therapeutically effective amount of an antibody, where the antibody comprises a VH region having a sequence at least 80%, 85%, 90%, 95%, 99%, or 100% identical to that of SEQ ID NO: 8 and a VL region having a sequence at least 80%, 85%, 90%, 95%, 99%, or 100% identical to that of SEQ ID NO: 9, where the antibody is present at an amount of 7500 mg as a unit dose, L-histidine present at 20 mM, methionine present at 5 mM, NaCl present at 100 mM, polysorbate 80 present at 0.02% and where the pH is about 5.8.

In some embodiments, the pharmaceutical antibody formulation is administered once per day, twice per day, three times per day or more. In some embodiments, the pharmaceutical antibody formulation is administered daily, every day, every alternate day, every ten days, five days a week, once a week, every other week, two weeks per month, three weeks per month, once a month, twice a month, three times per month, or more. The pharmaceutical antibody formulation is administered for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 2 years, 3 years, or more.

In the case wherein the patient's status does improve, upon the doctor's discretion, the administration of the pharmaceutical antibody formulation is given continuously; alternatively, the dose of the pharmaceutical antibody formulation being administered is temporarily reduced or temporarily suspended for a certain length of time (i.e., a “drug holiday”). In some embodiments, the length of the drug holiday varies between 2 days and 1 year. In some embodiments, the length of the drug holiday is 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days, or any length within a range defined by any two of the aforementioned times. The dose reduction during a drug holiday may be from 10%-100%. In some embodiments, the dose reduction during a drug holiday may be 10%, 15%, 20%, 25%, 30%, 35%, 40%, 45%, 50%, 55%, 60%, 65%, 70%, 75%, 80%, 85%, 90%, 95%, or 100%, or any percentage within a range defined by any two of the aforementioned percentages.

Once improvement of the patient's condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some embodiments, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in humans. The dosage of such compounds lies preferably within a range of circulating concentrations that include the ED50 with minimal toxicity. The dosage varies within this range depending upon the dosage form employed and the route of administration utilized.

As applied to any of the methods disclosed herein, in some embodiments, the pharmaceutical antibody formulation is administered enterally, orally, intranasally, parenterally, intracranially, subcutaneously, intramuscularly, intradermally, or intravenously, or any combination thereof. In some embodiments, the pharmaceutical antibody formulation is administered intravenously or subcutaneously. In some embodiments, the subject is a mammal. In some embodiments, the subject is a human.

In some embodiments, any of the pharmaceutical antibody compositions provided herein are used in a method for treating Alzheimer's disease. In some embodiments, the methods comprise administering any of the pharmaceutical antibody compositions disclosed herein to a subject in need of Alzheimer's disease treatment. In some embodiments, the methods further comprise detecting an improvement in the Alzheimer's disease in the subject after administration. In some embodiments, the pharmaceutical antibody formulation is administered daily, weekly, bi-weekly, or every 10 days. In some embodiments, the pharmaceutical antibody composition comprises a therapeutically effective amount of an antibody. In some embodiments, the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, the pharmaceutical antibody composition further comprises histidine, methionine, NaCl, and polysorbate, and the formulation is at a pH between 5.3 and 6.3. In some embodiments, the pharmaceutical antibody formulation further comprises sucrose, mannitol, or both. In some embodiments, the subject is administered 70 mg, 75 mg, 140 mg, 200 mg, 420 mg, 450 mg, 700 mg, 1500 mg, 2100 mg, 3750 mg, 5000 mg, or 7500 mg of antibody as a unit dose, or any amount of antibody as a unit dose within a range defined by any two of the aforementioned amounts. In some embodiments, the subject is administered 70 mg of antibody as a unit dose. In some embodiments, the subject is administered 75 mg of antibody as a unit dose. In some embodiments, the subject is administered 140 mg of antibody as a unit dose. In some embodiments, the subject is administered 200 mg of antibody as a unit dose. In some embodiments, the subject is administered 420 mg of antibody as a unit dose. In some embodiments, the subject is administered 450 mg of antibody as a unit dose. In some embodiments, the subject is administered 700 mg of antibody as a unit dose. In some embodiments, the subject is administered 1500 mg of antibody as a unit dose. In some embodiments, the subject is administered 2100 mg of antibody as a unit dose. In some embodiments, the subject is administered 3750 mg of antibody as a unit dose. In some embodiments, the subject is administered 5000 mg of antibody as a unit dose. In some embodiments, the subject is administered 7500 mg of antibody as a unit dose. In some embodiments, the unit dose is administered over the course of 10, 20, 30, 40, 50, 60, 70, 80, 90, 100, 120, 130, 140, 150, 160, 170, 180, 190, or 200 minutes, or any time within a range defined by any two of the aforementioned times. In some embodiments, the unit dose is administered over the course of 60 minutes. In some embodiments, the pharmaceutical antibody formulation is first diluted prior to administration such that the antibody is at a concentration of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations. In some embodiments, the pharmaceutical antibody formulation is first diluted prior to administration such that the pharmaceutical antibody formulation is administered in a volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes.

Also disclosed herein are methods of treating Alzheimer's disease. In some embodiments, the methods comprise administering a pharmaceutical antibody formulation to a subject in need of Alzheimer's disease treatment. In some embodiments, the pharmaceutical antibody formulation comprises a therapeutically effective amount of the antibody, where the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7. In some embodiments, the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg. In some embodiments, the pharmaceutical antibody formulation further comprises L-histidine present at 20 mM, methionine present at 5 mM, NaCl present at 100 mM, and polysorbate-80 present at 0.02%. In some embodiments, the pharmaceutical antibody formulation is at a pH of about 5.8.

In some embodiments, the methods further comprise identifying the subject in need of Alzheimer's disease treatment prior to administration, such as having Alzheimer's disease or at risk of contracting Alzheimer's disease. In some embodiments, the step of identifying the subject as in need of Alzheimer's disease treatment may be done according to the following inclusion criteria:
1) Can be ambulatory.
2) Can have a clinical diagnosis of Alzheimer's disease consistent with the following:
a) Probable Alzheimer's disease according to National Institute of Neurological and Communicative Disease and Stroke and Alzheimer's Disease and Related Disorders Association (NINCDS-ADRDA)
b) Dementia of Alzheimer's type, according to the Diagnostic and Statistical Manual of Mental Disorders (DSM) criteria.
3) Subject or caregiver has the ability to understand the purpose and risks of the study and provide signed and dated informed consent (or assent).
4) Can have a Mini Mental State Examination (MMSE) score of 14 to 26 (inclusive).

In some embodiments, the step of identifying the subject in need of Alzheimer's disease treatment comprises one or more of identifying probable Alzheimer's disease in the subject, identifying dementia of Alzheimer's disease type in the subject, or determining the subject as having a Mini Mental State Examination (MMSE) score of 14 to 26, inclusive. In some embodiments, the step of identifying the subject in need of Alzheimer's disease treatment comprises measuring plasma and cerebrospinal Aβ40, phosphorylated tau, neurofilament light chain protein (NfL), neurofilament heavy chain protein (NfH), or Gal3, determining the MMSE score of the subject, determining the neurocognitive fragility index (NFI) of the subject, observing brain atrophy in the subject, or observing amyloid plaques in the subject, or any combination thereof. In some embodiments, the treating step is to a patient that already has symptoms of Alzheimer's disease. In some embodiments, the treating step is prophylactic. In some embodiments, the methods for treating Alzheimer's disease further comprise monitoring the subject for an improvement in the Alzheimer's disease following the administering step. In some embodiments, monitoring the subject comprises measuring plasma and cerebrospinal fluid Aβ40, phosphorylated tau, neurofilament light chain protein (NfL), neurofilament heavy chain protein (NfH), or Gal3, determining an improvement in the MMSE score of the subject, determining an improvement in the neurocognitive fragility index (NFI) of the subject, observing a reduction in brain atrophy in the subject, or observing a reduction in amyloid plaques in the subject, or any combination thereof. Exemplary baseline levels for identifying a subject in need of Alzheimer's disease treatment and/or monitoring the subject for an improvement in the Alzheimer's disease following the administering step may include measuring levels of biomarkers as shown in Table 1, or observing an improvement in the levels of biomarkers as shown in Table 1.

### Polynucleotides and Vectors

In some embodiments, the present disclosure provides isolated nucleic acids encoding any of the anti-Gal3 antibodies used for the pharmaceutical antibody compositions disclosed herein. In another embodiment, the present disclosure provides vectors comprising a nucleic acid sequence encoding any anti-Gal3 antibody used for the pharmaceutical antibody compositions disclosed herein. In some embodiments, this disclosure provides isolated nucleic acids that encode heavy chain CDRs, light chain CDRs, heavy chain variable regions, light chain variable regions, heavy chains, or light chains of an anti-Gal3 antibody.

In some embodiments, an exemplary nucleic acid sequence encoding for a heavy chain variable region of an anti-Gal3 antibody is represented as SEQ ID NO: 12 or a sequence having at least 80%, 85%, 90%, 95%, 99%, or 100% identity to SEQ ID NO: 12. In some embodiments, an exemplary nucleic acid sequence encoding for a light chain variable region of an anti-Gal3 antibody is represented as SEQ ID NO: 13 or a sequence having at least 80%, 85%, 90%, 95%, 99%, or 100% identity to SEQ ID NO: 13. In some embodiments, an exemplary nucleic acid sequence encoding for a heavy chain of an anti-Gal3 antibody is represented as SEQ ID NO: 14 or a sequence having at least 80%, 85%, 90%, 95%, 99%, or 100% identity to SEQ ID NO: 14. In some embodiments, an exemplary nucleic acid sequence encoding for a light chain of an anti-Gal3 antibody is represented as SEQ ID NO: 15 or a sequence having at least 80%, 85%, 90%, 95%, 99%, or 100% identity to SEQ ID NO: 15. These exemplary nucleic acid sequences are depicted in FIG. 2C. It is envisioned that these nucleic acid sequences can be modified to result in the same or similar peptide sequence by virtue of codon swapping. The % identity of two sequences is well understood in the art and can be calculated by the number of conserved amino acids or nucleotides relative to the length of the sequences.

Any one of the anti-Gal3 antibodies described herein can be prepared by recombinant DNA technology, synthetic chemistry techniques, or a combination thereof. For instance, sequences encoding the desired components of the anti-Gal3 antibodies, including light chain CDRs and heavy chain CDRs are typically assembled cloned into an expression vector using standard molecular techniques known in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-Gal3 antibody of interest or binding fragment thereof.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A. 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-Gal3 antibodies can also be modified, for example, by substituting the coding sequence for human heavy and light chain constant regions in place of the homologous non-human sequences. In that manner, chimeric antibodies are prepared that retain the binding specificity of the original anti-Gal3 antibody.

### Exemplary Antibody Production

In some embodiments, anti-Gal3 antibodies or binding fragments thereof are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-Gal3 antibodies or binding fragments thereof can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, each incorporated fully herein by reference in its entirety. Such antibodies can be referred to as human xenogeneic antibodies.

Alternatively, anti-Gal3 antibodies or binding fragments thereof can be produced from phage libraries containing human variable regions. See U.S. Pat. No. 6,174,708, incorporated fully herein by reference in its entirety.

In some aspects of any of the embodiments disclosed herein, an anti-Gal3 antibody or binding fragment thereof is produced by a hybridoma.

For monoclonal anti-Gal3 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-Gal3 antibody or binding fragment thereof may be produced by genetic engineering.

Anti-Gal3 antibodies or binding fragments thereof disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-Gal3 antibodies or binding fragments thereof include, but are not limited to, humanized, chimeric, or xenogeneic human anti-Gal3 antibodies or binding fragments thereof.

Chimeric anti-Gal3 antibodies or binding fragments thereof can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some embodiments, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some embodiments, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains and incorporate only the complementarity-determining regions of the animal-derived antibody. This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755, each hereby incorporated by reference in its entirety.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

In some embodiments, the anti-Gal3 antibody or binding fragment thereof is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Biclonics, Fab-scFv-Fc, Two-in-one/Dual Action Fab (DAF), FinomAb, scFv-Fc-(Fab)-fusion, Dock-aNd-Lock (DNL), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some embodiments, the anti-Gal3 antibody or binding fragment thereof is a bispecific antibody or binding fragment thereof comprising a bispecific antibody format illustrated in Brinkmann and Kontermann, “The making of bispecific antibodies,” MABS 9(2): 182-212 (2017).

In some embodiments, the anti-Gal3 antibody or binding fragment thereof can comprise an IgM, IgG (e.g., IgG1, IgG2, IgG3, or IgG4), IgA, or IgE framework. The IgG framework can be IgG1, IgG2, IgG3 or IgG4. In some embodiments, the anti-Gal3 antibody or binding fragment thereof comprises an IgG1 framework. In some embodiments, the anti-Gal3 antibody or binding fragment thereof comprises an IgG2 framework. In some embodiments, the anti-Gal3 antibody or binding fragment thereof comprises an IgG4 framework. The anti-Gal3 antibody or binding fragment thereof can further comprise a Fc mutation.

In some embodiments, the Fc region comprises one or more mutations that modulate Fc receptor interactions, e.g., to enhance effector functions such as ADCC and/or CDC. In such instances, exemplary residues when mutated modulate effector functions include S239, K326, A330, 1332, or E333, in which the residue position correspond to IgG1 and the residue numbering is in accordance to Kabat numbering (EU index of Kabat et al 1991 Sequences of Proteins of Immunological Interest). In some embodiments, the one or more mutations comprise S239D, K326W, A330L, 1332E, E333A, E333S, or a combination thereof. In some embodiments, the one or more mutations comprise S239D, 1332E, or a combination thereof. In some embodiments, the one or more mutations comprise S239D, A330L, 1332E, or a combination thereof. In some embodiments, the one or more mutations comprise K326W, E333S, or a combination thereof. In some embodiments, the mutation comprises E333A.

In some embodiments, an anti-Gal3 antibody or binding fragment thereof can be either “monospecific” or “multi-specific”. Multi-specific anti-Gal3 antibodies or binding fragments thereof can be further classified on the basis of their binding specificities. A “monospecific” anti-Gal3 antibody or binding fragment thereof is a molecule capable of binding to one or more antigens of the same kind. A “multi-specific” anti-Gal3 antibody or binding fragment thereof is a molecule having binding specificities for at least two different antigens. While such molecules normally will only bind two distinct antigens (i.e. bispecific anti-Gal3 antibodies), antibodies with additional specificities (e.g. tri-specific, tetra-specific, and so on) are encompassed by this expression when used herein. This disclosure further provides multi-specific anti-Gal3 antibodies. Multi-specific anti-Gal3 antibodies or binding fragments thereof are multi-specific molecules capable of binding to at least two distinct antigens, e.g., bispecific and tri-specific molecules exhibiting binding specificities to two and three distinct antigens, respectively, where at least one antigen is not Gal3 or any portion, fragment, derivative, or modification thereof.

### Payload

In some embodiments, any of the pharmaceutical antibody compositions comprise an anti-Gal3 antibody that further comprises a payload. In some embodiments, the payload comprises a small molecule, a protein or functional fragment thereof, a peptide, or a nucleic acid polymer.

In some embodiments, the number of payloads conjugated to the anti-Gal3 antibody (e.g., the drug-to-antibody ratio or DAR) is about 1:1, one payload to one anti-Gal3 antibody. In some embodiments, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the ratio of the payloads to the anti-Gal3 antibody is about 2:1. In some embodiments, the ratio of the payloads to the anti-Gal3 antibody is about 3:1. In some embodiments, the ratio of the payloads to the anti-Gal3 antibody is about 4:1. In some embodiments, the ratio of the payloads to the anti-Gal3 antibody is about 6:1. In some embodiments, the ratio of the payloads to the anti-Gal3 antibody is about 8:1. In some embodiments, the ratio of the payloads to the anti-Gal3 antibody is about 12:1.

In some embodiment, the payload is a small molecule. In some embodiments, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some embodiments, the DNA cleaver comprises bleomycin A2, calicheamicin, or derivatives or analogs thereof. In some embodiments, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some embodiments, the DNA transcription inhibitor comprises dactinomycin. In some embodiments, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some embodiments, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵P, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-18F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, allophycocyanin (APC), phycoerythrin (PE), fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP) and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, 0-galactosidase, 0-glucoronidase or 0-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein or otherwise known in the art, such as those described in WO 2020/160156.

In some embodiments, the payload is an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG) molecule, or a combination of two or more of the agents. In some embodiments, the payload comprises a neuroactive polypeptide. In some embodiments, the neuroactive polypeptide is a neurotrophic factors, endocrine factors, growth factors, paracrine factors, hypothalamic release factors, neurotransmitter polypeptides, polypeptide agonists for a receptor expressed by a CNS cell, polypeptides involved in lysosomal storage disease or any combination thereof. In some embodiments, the payload is another antibody, or a heavy and/or light chain, or any other fragment thereof. In some embodiments, the payload comprises a heterologous antibody or fragment thereof.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapristone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovir, etanercept, tacrolimus, sirolimus, voclosporin, cyclosporine, rapamycin, cyclophosphamide, azathioprine, mycophenolate mofetil, methotrexate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some embodiments, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some embodiments, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some embodiments, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some embodiments, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some embodiments, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Conjugation Chemistry

In some embodiments, the payload is conjugated to an anti-Gal3 antibody described herein by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910.

In some embodiments, the payload is conjugated to an anti-Gal3 antibody described herein by a site-directed method utilizing a “traceless” coupling technology (Philochem). In some embodiments, the “traceless” coupling technology utilizes an N-terminal 1,2-aminothiol group on the binding moiety which is then conjugated with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some embodiments, the payload is conjugated to an anti-Gal3 antibody described herein by a site-directed method utilizing an unnatural amino acid incorporated into the binding moiety. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some embodiments, the payload is conjugated to an anti-Gal3 antibody described herein by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some embodiments, the enzyme-catalyzed process comprises microbial transglutaminase (mTG). In some embodiments, the payload is conjugated to the anti-Gal3 antibody utilizing a microbial transglutaminase catalyzed process. In some embodiments, mTG catalyzes the formation of a covalent bond between the amide side chain of a glutamine within the recognition sequence and a primary amine of a functionalized polynucleic acid molecule. In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, the payload is conjugated to an anti-Gal3 antibody by a method as described in PCT Publication No. WO2014/140317, which utilizes a sequence-specific transpeptidase and is hereby expressly incorporated by reference in its entirety.

In some embodiments, the payload is conjugated to an anti-Gal3 antibody described herein by a method as described in U.S. Patent Publication Nos. 2015/0105539 and 2015/0105540.

### Linker

In some embodiments, a linker described herein comprises a natural or synthetic polymer, consisting of long chains of branched or unbranched monomers, and/or cross-linked network of monomers in two or three dimensions. In some embodiments, the linker includes a polysaccharide, lignin, rubber, or polyalkylene oxide (e.g., polyethylene glycol).

In some embodiments, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some embodiments, block copolymers are polymers wherein at least one section of a polymer is built up from monomers of another polymer. In some embodiments, the linker comprises polyalkylene oxide. In some embodiments, the linker comprises PEG. In some embodiments, the linker comprises polyethylene imide (PEI) or hydroxy ethyl starch (HES).

In some embodiments, the polyalkylene oxide (e.g., PEG) is a polydisperse or monodisperse compound. In some embodiments, polydisperse material comprises disperse distribution of different molecular weight of the material, characterized by mean weight (weight average) size and dispersity. In some embodiments, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide unit. In some embodiments, a discrete PEG (dPEG) comprises from 2 to 60, from 2 to 50, or from 2 to 48 repeating ethylene oxide units. In some embodiments, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some embodiments, a dPEG comprises about 2 or more repeating ethylene oxide units. In some embodiments, a dPEG is synthesized as a single molecular weight compound from pure (e.g., about 95%, 98%, 99%, or 99.5%) starting material in a step-wise fashion. In some embodiments, a dPEG has a specific molecular weight, rather than an average molecular weight.

In some embodiments, the linker is a discrete PEG, optionally comprising from 2 to 60, from 2 to 50, or from 2 to 48 repeating ethylene oxide units. In some embodiments, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In some embodiments, the linker is a polypeptide linker. In some embodiments, the polypeptide linker comprises at least 2, 3, 4, 5, 6, 7, 8, 10, 15, 20, 25, 30, 35, 40, 45, 50, 60, 70, 80, 90, 100, or more amino acid residues. In some embodiments, the polypeptide linker comprises at least 2, 3, 4, 5, 6, 7, 8, or more amino acid residues. In some embodiments, the polypeptide linker comprises at most 2, 3, 4, 5, 6, 7, 8, or less amino acid residues. In some embodiments, the polypeptide linker is a cleavable polypeptide linker (e.g., either enzymatically or chemically). In some embodiments, the polypeptide linker is a non-cleavable polypeptide linker. In some embodiments, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some embodiments, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some embodiments, the polypeptide linker comprises L-amino acids, D-amino acids, or a mixture of both L- and D-amino acids.

In some embodiments, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(T-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (ρNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some embodiments, the linker comprises a val-cit-PABA group. In additional cases, the linker comprises a mc-val-cit-PABA group.

In some embodiments, the linker is a self-immolative linker or a self-elimination linker. In some embodiments, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO2015038426.

In some embodiments, the linker is a dendritic type linker. In some embodiments, the dendritic type linker comprises a branching, multifunctional linker moiety. In some embodiments, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783.

The invention(s) are generally disclosed herein using affirmative language to describe the numerous embodiments. The invention also includes embodiments in which subject matter is excluded, in full or in part, such as substances or materials, method steps and conditions, protocols, or procedures.

Some embodiments provided herein are described by way of the following provided numbered arrangements and also provided as possible combinations or overlapping embodiments:

1. A pharmaceutical antibody formulation comprising:
a therapeutically effective amount of an antibody, wherein the antibody comprises a heavy chain CDR1 (HCDR1) having the sequence of SEQ ID NO: 2, a heavy chain CDR2 (HCDR2) having the sequence of SEQ ID NO: 3, a heavy chain CDR3 (HCDR3) having the sequence of SEQ ID NO: 4, a light chain CDR1 (LCDR1) having the sequence of SEQ ID NO: 5, a light chain CDR2 (LCDR2) having the sequence of SEQ ID NO: 6; and a light chain CDR3 (LCDR3) having the sequence of SEQ ID NO: 7;
histidine;
methionine;
NaCl; and
polysorbate, wherein the formulation is at a pH between 5.3 and 6.3.

2. The pharmaceutical antibody formulation of arrangement 1, wherein the histidine is L-histidine.

3. The pharmaceutical antibody formulation of arrangement 2, wherein the L-histidine is present at 10 to 50 mM.

4. The pharmaceutical antibody formulation of arrangement 2, wherein the L-histidine is present at about 20 mM.

5. The pharmaceutical antibody formulation of any one of the preceding arrangements, wherein the methionine is present at 2 to 10 mM.

6. The pharmaceutical antibody formulation of any one of the preceding arrangements, wherein the methionine is present at 5 mM.

7. The pharmaceutical antibody formulation of any one of the preceding arrangements, wherein the NaCl is present at 50 to 150 mM.

8. The pharmaceutical antibody formulation of any one of the preceding arrangements, wherein the NaCl is present at 100 mM.

9. The pharmaceutical antibody formulation of any one of the preceding arrangements, wherein the polysorbate comprises polysorbate-20, polysorbate-40, polysorbate-60, polysorbate-80, or any combination thereof.

10. The pharmaceutical antibody formulation of any one of the preceding arrangements, wherein the polysorbate comprises polysorbate-80.

11. The pharmaceutical antibody formulation of arrangement 10, wherein the polysorbate 80 is present at 0.01 to 0.04% or about 0.01% to about 0.04%.

12. The pharmaceutical antibody formulation of arrangement 11, wherein the polysorbate 80 is present at 0.02% or about 0.02%.

13. The pharmaceutical antibody formulation of any one of the preceding arrangements, wherein the pH is about 5.8.

14. The pharmaceutical antibody formulation of any one of the preceding arrangements, wherein the pH is 5.8.

15. The pharmaceutical antibody formulation of any one of the preceding arrangements, further comprising sucrose, mannitol, or both.

16. The pharmaceutical antibody formulation of arrangement 15, wherein the sucrose is present at 2% to 5% or about 2% to about 5%.

17. The pharmaceutical antibody formulation of arrangement 15 or 16, wherein mannitol is present at 2% to 5% or about 2% to about 5%.

18. The pharmaceutical antibody formulation of any one of the preceding arrangements, wherein the antibody is present at an amount of 70 to 7500 mg as a unit dose.

19. The pharmaceutical antibody formulation of any one of the preceding arrangements, wherein the antibody is present at an amount of one of: 70 mg, 75 mg, 140 mg, 200 mg, 420 mg, 450 mg, 700 mg, 1500 mg, 2100 mg, 3750 mg, 5000 mg, or 7500 mg as a unit dose.

20. The pharmaceutical antibody formulation of any one of the preceding arrangements, wherein the antibody is present at an amount of one of: 75 mg, 450 mg, 1500 mg, 3750 mg, or 7500 mg as a unit dose, or any amount within a range defined by any two of the aforementioned amounts.

21. The pharmaceutical antibody formulation of any one of the preceding arrangements, wherein the antibody is present at an amount of one of: 70 mg, 140 mg, 200 mg, 420 mg, 700 mg, 2100 mg, or 5000 mg as a unit dose, or any amount within a range defined by any two of the aforementioned amounts.

22. The pharmaceutical antibody formulation of any one of the preceding arrangements, wherein the antibody is present at a concentration of one of: 1 mg/mL, 5 mg/mL, 10 mg/mL, 20 mg/mL, 40 mg/mL, or 50 mg/mL, or any concentration within a range defined by any two of the aforementioned concentrations.

23. The pharmaceutical antibody formulation of any one of the preceding arrangements, wherein L-histidine is present at about 20 mM, methionine is present at about 5 mM, NaCl is present at about 100 mM, polysorbate 80 is present at about 0.02%, sucrose is present at 2-5%, mannitol is present at 2-5%, the pH is about 5.8, and wherein the therapeutically effective amount of the antibody is one of: 70 mg, 75 mg, 140 mg, 200 mg, 420 mg, 450 mg, 700 mg, 1500 mg, 2100 mg, 3750 mg, 5000 mg, or 7500 mg as a unit dose, or any amount within a range defined by any two of the aforementioned amounts.

24. The pharmaceutical antibody formulation of arrangement 23, wherein the therapeutically effective amount of the antibody is one of: 75 mg, 450 mg, 1500 mg, 3750 mg, or 7500 mg as a unit dose.

25. The pharmaceutical antibody formulation of arrangement 23, wherein the therapeutically effective amount of the antibody is one of: 70 mg, 140 mg, 200 mg, 420 mg, 700 mg, 2100 mg, or 5000 mg as a unit dose.

26. A pharmaceutical antibody formulation comprising:
a therapeutically effective amount of an antibody,
wherein the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, and wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg;
L-histidine is present at 20 mM;
methionine is present at 5 mM;
NaCl is present at 100 mM;
polysorbate 80 is present at 0.02%; and
the pH is about 5.8.

27. The pharmaceutical antibody formulation of arrangement 26, wherein the antibody is present at an amount as a unit dose of: 75 mg, 450 mg, 1500 mg, 3750 mg, or 7500 mg.

28. The pharmaceutical antibody formulation of arrangement 26, wherein the antibody is present in an amount as a unit dose of: 70 mg, 140 mg, 200 mg, 420 mg, 700 mg, 2100 mg, or 5000 mg.

29. The pharmaceutical antibody formulation of any one of the preceding arrangements, wherein sucrose is present at 2-5% and mannitol is present at 2-5%.

30. The pharmaceutical antibody formulation of any one of the preceding arrangements, wherein the formulation is configured for parenteral administration.

31. The pharmaceutical antibody formulation of any one of arrangements 1-30, wherein the formulation is configured for subcutaneous administration.

32. The pharmaceutical antibody formulation of arrangement 31, wherein the formulation configured for subcutaneous administration comprises sucrose or mannitol, or both.

33. The pharmaceutical antibody formulation of any one of arrangements 1-32, wherein the formulation is configured for intravenous administration.

34. The pharmaceutical antibody formulation of arrangement 33, wherein the formulation configured for intravenous administration does not comprise sucrose or mannitol, or both.

35. The pharmaceutical antibody formulation of any one of the preceding arrangements, wherein the pharmaceutical antibody formulation is prepared at a concentration of antibody of 20 mg/mL or 50 mg/mL.

36. The pharmaceutical antibody formulation of any one of the preceding arrangements, wherein the pharmaceutical antibody formulation remains 60% stable over 3 months at either 5° C. or 25° C./60% relative humidity (RH).

37. A pharmaceutical antibody formulation comprising:
a therapeutically effective amount of an antibody, wherein the antibody comprises a HCDR1 having the sequence of SEQ ID NO: 2, a HCDR2 having the sequence of SEQ ID NO: 3, a HCDR3 having the sequence of SEQ ID NO: 4, a LCDR1 having the sequence of SEQ ID NO: 5, a LCDR2 having the sequence of SEQ ID NO: 6; and a LCDR3 having the sequence of SEQ ID NO: 7, wherein each CDR can have up to 1, 2, 3, 4, or 5 amino acids changed from the recited sequence;
histidine;
methionine;
NaCl; and
polysorbate, wherein the formulation is at a pH between 5.3 and 6.3.

38. The pharmaceutical antibody formulation of arrangement 37, further comprising sucrose or mannitol, or both.

39. The pharmaceutical antibody formulation of arrangement 37 or 38, wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg.

40. The pharmaceutical antibody formulation of any one of arrangements 37-39, wherein the antibody is present at an amount as a unit dose of: 75 mg, 450 mg, 1500 mg, 3750 mg, or 7500 mg.

41. The pharmaceutical antibody formulation of any one of arrangements 37-40, wherein the antibody is present in an amount as a unit dose of: 70 mg, 140 mg, 200 mg, 420 mg, 700 mg, 2100 mg, or 5000 mg.

42. A sterile vial comprising a pharmaceutical antibody formulation, wherein the pharmaceutical antibody formulation comprises a therapeutically effective amount of an antibody, wherein the antibody comprises an HCDR1 having the sequence of SEQ ID NO; 2, an HCDR2 having the sequence of SEQ ID NO: 3, an HCDR3 having the sequence of SEQ ID NO; 4, an LCDR1 having the sequence of SEQ ID NO: 5, an LCDR2 having the sequence of SEQ ID NO: 6; and an LCDR3 having the sequence of SEQ ID NO: 7.

43. The sterile vial of arrangement 37, wherein the pharmaceutical antibody formation further comprises histidine, methionine, NaCl, and polysorbate, and wherein the formulation is at a pH between 5.3 and 6.3.

44. The sterile vial of arrangement 37 or 38, wherein the sterile vial is a 5 mL or 10 mL sterile vial.

45. The sterile vial of any one of arrangements 37-39, wherein the sterile vial contains 2, 3, 4, 5, 6, 7, 8, 9, or 10 mL of the pharmaceutical antibody formulation.

46. The sterile vial of any one of arrangements 37-40, wherein the sterile vial contains 2 mL or at least 2 mL of the pharmaceutical antibody formulation.

47. The sterile vial of any one of arrangements 37-40, wherein the sterile vial contains 8 mL or at least 8 mL of the pharmaceutical antibody formulation.

48. The sterile vial of any one of arrangements 37-42, wherein the pharmaceutical antibody formulation is a concentrated form of the pharmaceutical antibody formulation of any one of arrangements 1-36.

49. The sterile vial of arrangement 43, wherein the concentrated form of the pharmaceutical antibody formulation is at a concentration of 20, 30, 40, 50, 60, 70, 80, 90, or 100 mg/mL of antibody, or any concentration within a range defined by any two of the aforementioned concentrations.

50. The sterile vial of arrangement 43 or 44, wherein the concentrated form of the pharmaceutical antibody formulation is at a concentration of 20 mg/mL or at least 20 mg/mL of antibody.

51. The sterile vial of any one of arrangements 43-45, wherein the concentrated form of the pharmaceutical antibody formulation is at a concentration of 50 mg/mL or at least 50 mg/mL of antibody.

52. The sterile vial of any one of arrangements 43-46, wherein the concentrated form of the pharmaceutical antibody formulation is intended to be diluted 1×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, or 100× fold, or any fold within a range defined by any two of the aforementioned fold.

53. The sterile vial of any one of arrangements 43-47, wherein the concentrated form of the pharmaceutical antibody formulation is intended to be diluted to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations.

54. The sterile vial of any one of arrangements 43-48, wherein the concentrated form of the pharmaceutical antibody formulation is intended to be diluted into a final volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes.

55. The sterile vial of any one of arrangements 43-49, wherein the concentrated form of the pharmaceutical antibody formulation is intended to be diluted with saline.

56. The sterile vial of any one of arrangements 43-50, wherein the pharmaceutical antibody formulation is configured for parenteral administration.

57. The sterile vial of any one of arrangements 43-51, wherein the pharmaceutical antibody formulation is configured for subcutaneous administration.

58. The sterile vial of arrangement 52, wherein the pharmaceutical antibody formulation configured for subcutaneous administration comprises sucrose or mannitol, or both.

59. The sterile vial of any one of arrangements 43-51, wherein the pharmaceutical antibody formulation is configured for intravenous administration.

60. The sterile vial of arrangement 54, wherein the pharmaceutical antibody formulation configured for intravenous administration does not comprise sucrose or mannitol, or both.

61. The sterile vial of any one of arrangements 37-55, wherein the pharmaceutical antibody formulation remains 60% stable over 3 months at either 5° C. or 25° C./60% relative humidity (RH).

62. The pharmaceutical antibody formulation of any one of the preceding pharmaceutical antibody formulation arrangements or sterile vial arrangements, wherein the antibody comprises a heavy chain variable domain (VH) region having a sequence at least 80%, 85%, 90%, 95%, 99%, or 100% identical to that of SEQ ID NO: 8.

63. The pharmaceutical antibody formulation of any one of the preceding pharmaceutical antibody formulation arrangements or sterile vial arrangements, wherein the antibody comprises a light chain variable domain (VL) region having a sequence at least 80%, 85%, 90%, 95%, 99%, or 100% identical to that of SEQ ID NO: 9.

64. The pharmaceutical antibody formulation of any one of the preceding pharmaceutical antibody formulation arrangements or sterile vial arrangements, wherein the antibody comprises a VH region having a sequence at least 80%, 85%, 90%, 95%, 99%, or 100% identical to that of SEQ ID NO: 8, and wherein the antibody comprises a VL region having a sequence at least 80%, 85%, 90%, 95%, 99%, or 100% identical to that of SEQ ID NO: 9.

65. The pharmaceutical antibody formulation of any one of the preceding pharmaceutical antibody formulation arrangements or sterile vial arrangements, wherein the antibody comprises a VH region having a sequence of SEQ ID NO: 8.

66. The pharmaceutical antibody formulation of any one of the preceding pharmaceutical antibody formulation arrangements or sterile vial arrangements, wherein the antibody comprises a VL region having a sequence of SEQ ID NO: 9.

67. The pharmaceutical antibody formulation of any one of the preceding pharmaceutical antibody formulation arrangements or sterile vial arrangements, wherein the antibody comprises a VH region having a sequence of SEQ ID NO: 8, and wherein the antibody comprises a VL region having a sequence of SEQ ID NO: 9.

68. The pharmaceutical antibody formulation of any one of the preceding pharmaceutical antibody formulation arrangements or sterile vial arrangements, wherein the antibody comprises a heavy chain (HC) having a sequence at least 80%, 85%, 90%, 95%, 99%, or 100% identical to that of SEQ ID NO: 10.

69. The pharmaceutical antibody formulation of any one of the preceding pharmaceutical antibody formulation arrangements or sterile vial arrangements, wherein the antibody comprises a light chain (LC) having a sequence at least 80%, 85%, 90%, 95%, 99%, or 100% identical to that of SEQ ID NO: 11.

70. The pharmaceutical antibody formulation of any one of the preceding pharmaceutical antibody formulation arrangements or sterile vial arrangements, wherein the antibody comprises an HC having a sequence of SEQ ID NO: 10.

71. The pharmaceutical antibody formulation of any one of the preceding pharmaceutical antibody formulation arrangements or sterile vial arrangements, wherein the antibody comprises an LC having a sequence of SEQ ID NO: 11.

72. The pharmaceutical antibody formulation of any one of the preceding pharmaceutical antibody formulation arrangements or sterile vial arrangements, wherein the antibody comprises a VH that is encoded by a nucleic acid sequence having at least 80%, 85%, 90%, 95%, 99%, or 100% identical to that of SEQ ID NO: 12.

73. The pharmaceutical antibody formulation of any one of the preceding pharmaceutical antibody formulation arrangements or sterile vial arrangements, wherein the antibody comprises a VL that is encoded by a nucleic acid sequence having at least 80%, 85%, 90%, 95%, 99%, or 100% identical to that of SEQ ID NO: 13.

74. The pharmaceutical antibody formulation of any one of the preceding pharmaceutical antibody formulation arrangements or sterile vial arrangements, wherein the antibody comprises a VH that is encoded by a nucleic acid sequence of SEQ ID NO: 12.

75. The pharmaceutical antibody formulation of any one of the preceding pharmaceutical antibody formulation arrangements or sterile vial arrangements, wherein the antibody comprises a VL that is encoded by a nucleic acid sequence of SEQ ID NO: 13.

76. The pharmaceutical antibody formulation of any one of the preceding pharmaceutical antibody formulation arrangements or sterile vial arrangements, wherein the antibody comprises an HC that is encoded by a nucleic acid sequence having at least 80%, 85%, 90%, 95%, 99%, or 100% identical to that of SEQ ID NO: 14.

77. The pharmaceutical antibody formulation of any one of the preceding pharmaceutical antibody formulation arrangements or sterile vial arrangements, wherein the antibody comprises an LC that is encoded by a nucleic acid sequence having at least 80%, 85%, 90%, 95%, 99%, or 100% identical to that of SEQ ID NO: 15.

78. The pharmaceutical antibody formulation of any one of the preceding pharmaceutical antibody formulation arrangements or sterile vial arrangements, wherein the antibody comprises an HC that is encoded by a nucleic acid sequence of SEQ ID NO: 14.

79. The pharmaceutical antibody formulation of any one of the preceding pharmaceutical antibody formulation arrangements or sterile vial arrangements, wherein the antibody comprises an LC that is encoded by a nucleic acid sequence of SEQ ID NO: 15.

80. The pharmaceutical antibody formulation of any one of the preceding pharmaceutical antibody formulation arrangements or sterile vial arrangements, wherein the formulation exhibits a pH of between 5.7-5.9 or about 5.7-5.9 when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

81. The pharmaceutical antibody formulation of any one of the preceding pharmaceutical antibody formulation arrangements or sterile vial arrangements, wherein the formulation exhibits a monomeric purity of 97.5-99.7% or about 97.5-99.7% as determined by size exclusion chromatography when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

82. The pharmaceutical antibody formulation of any one of the preceding pharmaceutical antibody formulation arrangements or sterile vial arrangements, wherein the formulation exhibits a pI of 7.0 or about 7.0 as determined by capillary isoelectric focusing (cIEF) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

83. The pharmaceutical antibody formulation of any one of the preceding pharmaceutical antibody formulation arrangements or sterile vial arrangements, wherein the formulation exhibits a cIEF acidic peak of 15.0-54.7% or about 15.0-54.7% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

84. The pharmaceutical antibody formulation of any one of the preceding pharmaceutical antibody formulation arrangements or sterile vial arrangements, wherein the formulation exhibits a cIEF main peak of 39.7-78.8% or about 39.7-78.8% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

85. The pharmaceutical antibody formulation of any one of the preceding pharmaceutical antibody formulation arrangements or sterile vial arrangements, wherein the formulation exhibits a cIEF basic peak of 5.5-8.9% or about 5.5-8.9% when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

86. The pharmaceutical antibody formulation of any one of the preceding pharmaceutical antibody formulation arrangements or sterile vial arrangements, wherein the formulation exhibits a peak of non-reduced (NR) monomer of 98.1-100% or about 98.1-100% as determined by capillary electrophoresis (CE) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

87. The pharmaceutical antibody formulation of any one of the preceding pharmaceutical antibody formulation arrangements or sterile vial arrangements, wherein the formulation exhibits a peak of reduced (R) heavy chain and light chain (HC+LC) of 97.8-100% or about 97.8-100% as determined by CE when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

88. The pharmaceutical antibody formulation of any one of the preceding pharmaceutical antibody formulation arrangements or sterile vial arrangements, wherein the formulation exhibits a dissociation constant (KD) of 1.7-4.2 or about 1.7-4.2 as determined by biolayer interferometry (BLI) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

89. The pharmaceutical antibody formulation of any one of the preceding pharmaceutical antibody formulation arrangements or sterile vial arrangements, wherein the formulation exhibits a pI of 7.0 or about 7.0 as determined by cIEF when stored at a) 5° C. for 1, 3, 6, 9, 12, or 18 months, or b) 25° C. for 1, 3, or 6 months.

90. The pharmaceutical antibody formulation of any one of the preceding pharmaceutical antibody formulation arrangements or sterile vial arrangements, wherein the formulation exhibits a cIEF acidic peak of 17-26% or about 17-26% when stored at a) 5° C. for 1, 3, 6, 9, 12, or 18 months, or b) 25° C. for 1, 3, or 6 months.

91. The pharmaceutical antibody formulation of any one of the preceding pharmaceutical antibody formulation arrangements or sterile vial arrangements, wherein the formulation exhibits a cIEF main peak of 66-77% or about 66-7% when stored at a) 5° C. for 1, 3, 6, 9, 12, or 18 months, or b) 25° C. for 1, 3, or 6 months.

92. The pharmaceutical antibody formulation of any one of the preceding pharmaceutical antibody formulation arrangements or sterile vial arrangements, wherein the formulation exhibits a cIEF basic peak of 6-8% or about 6-8% when stored at a) 5° C. for 1, 3, 6, 9, 12, or 18 months, or b) 25° C. for 1, 3, or 6 months.

93. The pharmaceutical antibody formulation of any one of the preceding pharmaceutical antibody formulation arrangements or sterile vial arrangements, wherein the formulation exhibits a monomeric purity of 99.3-99.5% or about 99.3-99.5% as determined by ultra-high performance liquid chromatography-size exclusion chromatography (UPLC-SEC) when stored at a) 5° C. for 1, 3, 6, 9, 12, or 18 months, or b) 25° C. for 1, 3, or 6 months.

94. The pharmaceutical antibody formulation of any one of the preceding pharmaceutical antibody formulation arrangements or sterile vial arrangements, wherein the formulation exhibits a peak of non-reduced (NR) monomer of 98-99% or about 98-99% as determined by CE when stored at a) 5° C. for 1, 3, 6, 9, 12, or 18 months, or b) 25° C. for 1, 3, or 6 months.

95. The pharmaceutical antibody formulation of any one of the preceding pharmaceutical antibody formulation arrangements or sterile vial arrangements, wherein the formulation exhibits a peak of reduced (R) heavy chain and light chain (HC+LC) of 99.1-100% or about 99.1-100% as determined by CE when stored at a) 5° C. for 1, 3, 6, 9, 12, or 18 months, or b) 25° C. for 1, 3, or 6 months.

96. The pharmaceutical antibody formulation of any one of the preceding pharmaceutical antibody formulation arrangements or sterile vial arrangements, wherein the formulation exhibits a dissociation constant (KD) of 2.0-3.7 nM or about 2.0-3.7 nM as determined by biolayer interferometry (BLI) when stored at a) 5° C. for 1, 3, 6, 9, 12, or 18 months, or b) 25° C. for 1, 3, or 6 months.

97. The pharmaceutical antibody formulation of any one of the preceding pharmaceutical antibody formulation arrangements or sterile vial arrangements, wherein the formulation exhibits an IC50 of 1.2-2.5 μg/mL or about 1.2-2.5 μg/mL as determined by ELISA when stored at a) 5° C. for 1, 3, 6, 9, 12, or 18 months, or b) 25° C. for 1, 3, or 6 months.

98. The pharmaceutical antibody formulation of any one of the preceding pharmaceutical antibody formulation arrangements or sterile vial arrangements, wherein the formulation exhibits a pH of 5.8-5.9 or about 5.8-5.9 when stored at a) 5° C. for 1, 3, 6, 9, 12, or 18 months, or b) 25° C. for 1, 3, or 6 months.

99. The pharmaceutical antibody formulation of any one of the preceding pharmaceutical antibody formulation arrangements or sterile vial arrangements, wherein the formulation comprises an appearance of a clear, colorless solution essentially free of particles when stored at a) 5° C. for 1, 3, 6, 9, 12, or 18 months, or b) 25° C. for 1, 3, or 6 months.

100. The pharmaceutical antibody formulation of any one of the preceding pharmaceutical antibody formulation arrangements or sterile vial arrangements, wherein the formulation exhibits an osmolality of 226-231 mOsm/kg or about 226-231 mOsm/kg when stored at a) 5° C. for 1, 3, 6, 9, 12, or 18 months, or b) 25° C. for 1, 3, or 6 months.

101. The pharmaceutical antibody formulation of any one of the preceding pharmaceutical antibody formulation arrangements or sterile vial arrangements, wherein the formulation remains sterile and/or free of bacterial endotoxins when stored at a) 5° C. for 1, 3, 6, 9, 12, or 18 months, or b) 25° C. for 1, 3, or 6 months.

102. A method of treating Alzheimer's disease, the method comprising: administering the pharmaceutical antibody formulation of any one of the preceding pharmaceutical antibody formulation arrangements or sterile vial arrangements to a subject in need of Alzheimer's disease treatment.

103. The method of arrangement 102, wherein the pharmaceutical antibody formulation is administered daily, weekly, bi-weekly, or every 10 days.

104. The method of arrangement 102 or 103, wherein the subject is administered 70 mg, 75 mg, 140 mg, 200 mg, 420 mg, 450 mg, 700 mg, 1500 mg, 2100 mg, 3750 mg, 5000 mg, or 7500 mg of antibody as a unit dose, or any amount of antibody as a unit dose within a range defined by any two of the aforementioned amounts.

105. The method of any one of arrangements 102-104, wherein the subject is administered a pharmaceutical antibody formulation comprising:
a therapeutically effective amount of the antibody,
wherein the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, and wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg;
L-histidine is present at 20 mM;
methionine is present at 5 mM;
NaCl is present at 100 mM;
polysorbate 80 is present at 0.02%; and
the pH is about 5.8.

106. The method of any one of arrangements 102-105, wherein the subject is administered 75 mg, 450 mg, 1500 mg, 3750 mg, or 7500 mg of antibody as a unit dose.

107. The method of any one of arrangements 102-105, wherein the subject is administered 70 mg, 140 mg, 200 mg, 420 mg, 700 mg, 2100 mg, or 5000 mg of antibody as a unit dose.

108. The method of any one of arrangements 102-107, wherein the unit dose is administered over the course of 10, 20, 30, 40, 50, 60, 70, 80, 90, 100, 120, 130, 140, 150, 160, 170, 180, 190, or 200 minutes.

109. The method of any one of arrangements 102-108, wherein the unit dose is administered over the course of 60 minutes.

110. The method of any one of arrangements 102-109, wherein the pharmaceutical antibody formulation is first diluted prior to administration such that the antibody is at a concentration of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations.

111. The method of any one of arrangements 102-110, wherein the pharmaceutical antibody formulation is first diluted prior to administration such that the pharmaceutical antibody formulation is administered in a volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes.

112. The method of arrangements 110 or 111, wherein the pharmaceutical antibody formulation is diluted in saline.

113. The method of any one of arrangements 102-112, further comprising a step of identifying a subject in need of Alzheimer's disease treatment.

114. The method of arrangement 113, wherein the step of identifying the subject in need of Alzheimer's disease treatment comprises one or more of identifying probable Alzheimer's disease in the subject, identifying dementia of Alzheimer's disease type in the subject, or determining the subject as having a Mini Mental State Examination (MMSE) score of 14 to 26, inclusive.

115. The method of one of arrangements 102-114, wherein the treating step is to a patient that already has symptoms of Alzheimer's disease.

116. The method of one of arrangements 102-115, wherein the treating step is prophylactic.

117. The method of any one of arrangements 102-116, further comprising monitoring the subject for an improvement in the Alzheimer's disease following the administering step.

118. The method of arrangement 117, wherein monitoring the subject comprises measuring plasma and cerebrospinal fluid Aβ40, phosphorylated tau, neurofilament light chain protein (NfL), neurofilament heavy chain protein (NfH), or Gal3, determining an improvement in the MMSE score of the subject, determining an improvement in the neurocognitive fragility index (NFI) of the subject, observing a reduction in brain atrophy in the subject, or observing a reduction in amyloid plaques in the subject, or any combination thereof.

119. The method of any one of arrangements 102-118, wherein the pharmaceutical antibody formulation is administered for 10-18 months.

120. The method of any one of arrangements 102-119, wherein the pharmaceutical antibody formulation is administered intravenously.

121. The method of any one of arrangements 102-120, wherein the pharmaceutical antibody formulation is administered subcutaneously.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Formulations of Anti-Gal3 Antibodies can be Used to Treat Alzheimer's Disease in Humans

A patient presents with Alzheimer's disease, is experiencing initial symptoms of Alzheimer's disease, or is at risk of developing Alzheimer's disease. One or more of the pharmaceutical antibody formulations disclosed herein are administered to the patient enterally, orally, intranasally, parenterally, intracranially, subcutaneously, intramuscularly, intradermally, or intravenously. In some embodiments, the formulation is administered intravenously or subcutaneously.

The pharmaceutical antibody formulation is administered to the patient such that 70 mg (or in the alternative: 75 mg, 140 mg, 200 mg, 420 mg, 450 mg, 700 mg, 1500 mg, 2100 mg, 3750 mg, 5000 mg, or 7500 mg) as a unit dose is administered to the patient. The unit dose of the formulation is administered over the duration of 60 minutes (or in the alternative: 10, 20, 30, 40, 50, 70, 80, 90, 100, 110, 120, 130, 140, 150, 160, 170, 180, 190, or 200 minutes). The formulation is administered daily (or in the alternative: weekly, bi-weekly, or every 10 days) for a duration of 10 months (or in the alternative: 11, 12, 13, 14, 15, 16, 17, or 18 months). Administration of the formulation may be performed in conjunction with another therapy for Alzheimer's disease, for example, a cholinesterase inhibitor (e.g. tacrine, rivastigmine, galantamine, donepezil), an NMDA receptor antagonist (e.g. memantine), or both.

An improvement of the Alzheimer's disease or symptoms associated with the Alzheimer's disease is observed in the patient following administration of the pharmaceutical antibody formulation. To monitor disease progression, diagnostics generally known in the art may be used. Patients are monitored for their plasma and cerebrospinal fluid Aβ40, phosphorylated tau, neurofilament light chain protein (NfL), neurofilament heavy chain protein (NfH), and Gal-3, their MMSE score, their neurocognitive frailty index (NFI), brain atrophy measured by volumetric measurements (e.g. by MRI), and presence of amyloid plaques (e.g. by MRI). Exemplary baseline levels for these characteristics in Alzheimer's disease patients is provided in Table 1. These baseline levels are observations found in different patient cohorts, and it is envisioned that one skilled in the art will recognize variations in these levels in other Alzheimer's disease patient populations. Improvements of the Alzheimer's disease or symptoms may include improvements in one or more of these characteristics. Additional exemplary baseline level studies of Alzheimer's disease patients may be found in Sánchez-Valle et al. “Serum neurofilament light levels correlate with severity measures and neurodegeneration markers in autosomal dominant Alzheimer's disease” Alzheimers Res. Ther. (2018) 10:113, Lehmann et al. “Relevance of Aβ42/40 Ratio for Detection of Alzheimers Disease Pathology in Clinical Routine: The PLM_{R} Scale” Front. Aging Neurosci. (2018) 10:138, Hanon et al. “Plasma amyloid levels within the Alzheimer's process and correlations with central biomarkers” Alzheimer's and Dementia (2018) 14:858-868, Seppala et al. “Plasma Aβ42 and Aβ40 as markers of cognitive change in follow-up: a prospective, longitudinal, population-based cohort study” J. Neurol. Neurosurg. Psychiatry (2010) 81:1123-1127, and Baldeiras et al. “Addition of the Aβ42/40 ratio to the cerebrospinal fluid biomarker profile increases the predictive value for underlying Alzheimer's disease dementia in mild cognitive impairment” Alzheimer's Research & Therapy (2018) 10:33, each of which is hereby expressly incorporated by reference in its entirety.

TABLE 1
Exemplary biomarker levels in Alzheimer's disease patients.

| | Baseline levels | | |
|---|---|---|---|
| | for Alzheimer's | | |
| Biomarker | disease patients | Units | Comments |
| Aβ40 | 263 ± 80 | pg/ml | In plasma |
| Aβ40 | 200-500 | ng/ml | In cerebrospinal |
| | | | fluid (CSF), |
| | | | expected to be |
| | | | higher in control |
| | | | population |
| Phospho-Tau | 0.1-0.2 | ng/μl | In serum |
| Phospho-Tau | 153.34 ± 132.26 | ng/ml | In CSF |
| NfL | 30.87 ± 15.14 | pg/ml | In serum |
| NfL | 2123.19 ± 649.45 | ng/ml | In CSF |
| NfH | 0.5-1 | ng/ml | In serum |
| NfH | 2000-5000 | pg/ml | In CSF |
| Gal3 | 6.42 ± 2.51 | ng/ml | In serum |
| Gal3 | Between 5-7 | ng/ml | In CSF |
| Amyloid plaque | Presence | n/a | n/a |
| by MRI | | | |
| MMSE score | Between 20-12 | points | n/a |
| NFI score | 15 ± 5 | points | n/a |
| Brain atrophy | Hippocampal volume | n/a | n/a |
| | reduction by 15-30% | | |
| | relative to controls | | |

### Example 2. Stability Test of Antibody Formulations

Short term and long term stability of TB006 antibody formulations and preparations were assessed.

Two bags of TB006 diluted to 0.31 mg/mL in sterile bags pre-filled with 250 mL of 0.9% Sodium Chloride for injection, USP were tested. 4 mL of a 20 mg/mL antibody solution was used. The solutions were confirmed to be colorless and free of particulate matter before the experiment began and throughout the time course. Diluted samples were stored at 25±3° C. and sampled at 0, 0.5 hours, 1 hour, 2 hours, and 4 hours.

Preparations of dilute TB006 antibody formulations in bags ready for parenteral administration were stored at room temperature for 0-4 hours. After 0-4 hours, the preparation maintained at least 60% viability, and no visible particles were observed (Table 2).

TABLE 2
Injection In-use Stability Study Results (Bag 1 and 2)

| | | Bag #1 | | | | |
|---|---|---|---|---|---|---|
| | | 0 h | 0.5 h | 1 h | 2 h | 4 h |
| Appearance | Appearance | Colorless liquid, no visible particle | | | | |
| SoloVPE | Conc. | 0.28 | 0.30 | 0.30 | 0.30 | 0.30 |
| concentration | (mg/mL) | | | | | |
| SEC-HPLC | Monomer (%) | 99.3 | 99.4 | 99.3 | 99.2 | 99.4 |
| cIEF | pI | 7.0 | 7.0 | 7.0 | 7.0 | 7.0 |
| | Acidic peak (%) | 16-16.3 | 17.8-18 | 18-18.3 | 19.8-20 | 19.9-20 |
| | Main Peak (%) | 76.7-77 | 73-73.2 | 73 | 72 | 72.8-73 |
| | Basic Peak (%) | 7 | 9-9.1 | 8.7-9 | 8-8.2 | 7-7.4 |
| CE-SDS | CE NR | 100 | 100 | 100 | 100 | 100 |
| | monomer (%) | | | | | |
| | CE R | 100 | 100 | 100 | 100 | 100 |
| | HC + LC (%) | | | | | |
| ELISA | IC₅₀ (μg/mL) | 1.007 | 1.341 | 1.426 | 1.714 | 1.412 |
| Blocking | | | | | | |
| Assay | | | | | | |
| HIAC | 2 μM | 890 | 1404 | 2082 | 2010 | 2307 |
| Subvisible | 5 μM | 116 | 221 | 397 | 400 | 471 |
| Particles | 10 μM | 12 | 39 | 69 | 83 | 111 |
| | 25 μM | 0 | 1.1 | 0 | 1.1 | 4.4 |

| Bag #2 | | | | |
|---|---|---|---|---|
| 0 h | 0.5 h | 1 h | 2 h | 4 h |

| | | |
|---|---|---|
| Appearance | Appearance | Colorless liquid, no visible particle |

| | | | | | | |
|---|---|---|---|---|---|---|
| SoloVPE | Conc. | 0.31 | 0.30 | 0.30 | 0.30 | 0.30 |
| concentration | (mg/mL) | | | | | |
| SEC-HPLC | Monomer (%) | 99.2 | 99.3 | 99.3 | 99.4 | 99.4 |
| cIEF | pI | 7.0 | 7.0 | 7.0 | 7.0 | 7.0 |
| | Acidic peak (%) | 17.8-18 | 15-15.2 | 16-16.4 | 18.5-19 | 16.7-17 |
| | Main Peak (%) | 73.9-74 | 77 | 76-76.3 | 73-73.4 | 76-76.1 |
| | Basic Peak (%) | 8-8.3 | 7.8-8 | 7-7.3 | 8-8.1 | 7-7.2 |
| CE-SDS | CE NR | 100 | 100 | 100 | 100 | 100 |
| | monomer (%) | | | | | |
| | CE R | 100 | 100 | 100 | 100 | 100 |
| | HC + LC (%) | | | | | |
| ELISA | IC₅₀ (μg/mL) | 1.051 | 1.657 | 1.627 | 1.671 | 1.354 |
| Blocking | | | | | | |
| Assay | | | | | | |
| HIAC | 2 μM | 156 | 68 | 27 | 60 | 44 |
| Subvisible | 5 μM | 53 | 17 | 2 | 20 | 11 |
| Particles | 10 μM | 14 | 4 | 1 | 4 | 2 |
| | 25 μM | 0 | 0 | 0 | 1 | 0 |

TB006 antibody formulations were kept for 0-3 months at either 5° C. for long term stability assays, or at 25° C./60% relative humidity (RH) for accelerated stability assays, according to standard FDA conditions. Both the long term and accelerated condition formulations were found to be within acceptable range for various tested parameters (Tables 3 and 4).

TABLE 3
Long-term Stability Testing Results (5° C.)

| | | Acceptance | | Testing results (Batch No. 095I0720) | | | | | |
|---|---|---|---|---|---|---|---|---|---|
| Test Item | Method | Criteria | | 0 months | | 1 month | | 3 months | |
| pI | cIEF | pI: 7.0 ± 0.3 | | pI: 7.0 | | pI: 7.0 | | pI: 7.0 | |
| Charge | cIEF | Acidic peak: ≤25% | | Acidic peak: 18% | | Acidic peak: 22% | | Acidic peak: 19% | |
| Variant | | Main peak: ≥65% | | Main peak: 74% | | Main peak: 71% | | Main peak: 73% | |
| | | Basic peak: ≤20% | | Basic peak: 8% | | Basic peak: 7% | | Basic peak: 8% | |
| Purity | SEC-UPLC | Monomer: ≥90.0% | | Monomer: 99.4% | | Monomer: 99.4% | | Monomer: 99.4% | |
| | | Aggregate: ≤10.0% | | Aggregate: 0.6% | | Aggregate: 0.6% | | Aggregate: 0.6% | |
| | | Fragment: ≤10.0% | | | | | | | |
| | Non-reduced | Monomer: ≥90% | | Monomer: 99% | | Monomer: 99% | | Monomer: 99% | |
| | CE-SDS | Fragment: ≤10% | | Fragment: 1% | | Fragment: 1% | | Fragment: 1% | |
| | Reduced | LC + HC: ≥90.0% | | LC + HC: 100.0% | | LC + HC: 100.0% | | LC + HC: 100.0% | |
| | CE-SDS | NGHC: ≤10.0% | | | | | | | |
| Binding | BLI | 50%-150% | | 3.0 | nM | 2.5 | nM | 3.0 | nM |
| Activity | | kD: 3.0 nM | | | | | | | |
| Protein | UV | 20.0 ± 2 | mg/mL | 20.3 | mg/mL | 20.0 | mg/mL | 20.1 | mg/mL |
| Content | | | | | | | | | |
| Polysorbate | FLD-HPLC | 0.1-0.3 | mg/mL | 0.28 | mg/mL | 0.24 | mg/mL | 0.21 | mg/mL |
| 80 (PS80) | | | | | | | | | |
| Biological | ELISA | 50%-150% | | 2.5 | μg/mL | 1.8 | μg/mL | 1.5 | μg/mL |
| Activity | | IC50: 2.0 μg/mL | | | | | | | |
| pH | | 5.8 ± 0.5 | | 5.8 | | 5.8 | | 5.8 | |
| Container | USP Chap. | ≥2.0 | mL | 2.2 | mL | 2.2 | mL | 2.1 | mL |

| Content | 697 | | | | | | |
|---|---|---|---|---|---|---|---|
| Appearance | Visual | Clear, | | Clear, | | Clear, | Clear, |
| | Method | colorless to | | colorless, | | colorless, | colorless, |
| | | light yellow, | | essentially | | essentially | essentially |
| | | essentially | | free of | | free of | free of |
| | | free of | | particles | | particles | particles |
| | | particles | | | | | |
| Osmolality | Freezing | 230 ± 25 | | 228 | | 226 | 227 |
| | Point | mOsmol/kg | | mOsmol/kg | | mOsmol/kg | mOsmol/kg |
| | Depression | | | | | | |
| Sterility | USP Chap. | Complies with | | Pass | | N/A | N/A |
| | 71 | the test for | | | | | |
| | | sterility | | | | | |
| Bacterial | Kinetic- | ≤0.6 | EU/mg | <0.01 | EU/mg | N/A | N/A |

| | | | | | |
|---|---|---|---|---|---|
| Endotoxin | chromogenic | | | | |
| | assay | | | | |
| Sub-visible | Light | ≥10 μm: ≤6000 | ≥10 μm: 9 | N/A | ≥10 μm: 6 |
| Particles | Blockage | particles/vial | particles/vial | | particles/vial |
| | Method | ≥25 μm: ≤600 | ≥25 μm: <1 | | ≥25 μm: <1 |
| | | particles/vial | particle/vial | | particle/vial |

TABLE 4
Accelerated Stability Testing Results (25° C./60% RH)

| | | Acceptance | | Testing results (Batch No. 095I0720) | | | | | |
|---|---|---|---|---|---|---|---|---|---|
| Test Item | Method | Criteria | | 0 months | | 1 month | | 3 months | |
| pI | cIEF | pI: 7.0 ± 0.3 | | pI: 7.0 | | pI: 7.0 | | pI: 7.0 | |
| Charge | cIEF | Acidic peak: ≤25% | | Acidic peak: 18% | | Acidic peak: 22% | | Acidic peak: 23% | |
| Variant | | Main peak: ≥65% | | Main peak: 74% | | Main peak: 70% | | Main peak: 70% | |
| | | Basic peak: ≤20% | | Basic peak: 8% | | Basic peak: 8% | | Basic peak: 7% | |
| Purity | SEC-UPLC | Monomer: ≥90.0% | | Monomer: 99.4% | | Monomer: 99.4% | | Monomer: 99.3% | |
| | | Aggregate: ≤10.0% | | Aggregate: 0.6% | | Aggregate: 0.6% | | Aggregate: 0.7% | |
| | | Fragment: ≤10.0% | | | | | | | |
| | Non-reduced | Monomer: ≥90% | | Monomer: 99% | | Monomer: 99% | | Monomer: 99% | |
| | CE-SDS | Fragment: ≤10% | | Fragment: 1% | | Fragment: 1% | | Fragment: 1% | |
| | Reduced | LC + HC: ≥90.0% | | LC + HC: 100.0% | | LC + HC: 100.0% | | LC + HC: 100.0% | |
| | CE-SDS | NGHC: ≤10.0% | | | | | | | |
| Binding | BLI | 50%-150% | | 3.0 | nM | 2.5 | nM | 3.1 | nM |
| Activity | | kD: 3.0 nM | | | | | | | |
| Protein | UV | 20.0 ± 2 | mg/mL | 20.3 | mg/mL | 19.9 | mg/mL | 20.1 | mg/mL |
| Content | | | | | | | | | |
| Polysorbate | FLD-HPLC | 0.1-0.3 | mg/mL | 0.28 | mg/mL | 0.24 | mg/mL | 0.22 | mg/mL |
| 80 (PS80) | | | | | | | | | |

| Biological | ELISA | 50%-150% | 2.5 | μg/mL | 1.8 | μg/mL | 1.5 | μg/mL |
|---|---|---|---|---|---|---|---|---|
| Activity | | IC50: 2.0 μg/mL | | | | | | |
| pH | | 5.8 ± 0.5 | 5.8 | | 5.9 | | 5.9 | |
| Container | USP Chap. | ≥2.0 mL | 2.2 | mL | 2.1 | mL | 2.2 | mL |
| Content | 697 | | | | | | | |

| Appearance | Visual | Clear, | | Clear, | | Clear, | Clear, |
|---|---|---|---|---|---|---|---|
| | Method | colorless to | | colorless, | | colorless, | colorless, |
| | | light yellow, | | essentially | | essentially | essentially |
| | | essentially | | free of | | free of | free of |
| | | free of | | particles | | particles | particles |
| | | particles | | | | | |
| Osmolality | Freezing | 230 ± 25 | | 228 | | 229 | 231 |
| | Point | mOsmol/kg | | mOsmol/kg | | mOsmol/kg | mOsmol/kg |
| | Depression | | | | | | |
| Sterility | USP Chap. | Complies with | | Pass | | N/A | N/A |
| | 71 | the test for | | | | | |
| | | sterility | | | | | |
| Bacterial | Kinetic- | ≤0.6 | EU/mg | <0.01 | EU/mg | N/A | N/A |

| | | | | | |
|---|---|---|---|---|---|
| Endotoxin | chromogenic | | | | |
| | assay | | | | |
| Sub-visible | Light | ≥10 μm: ≤6000 | ≥10 μm: 9 | N/A | ≥10 μm: 7 |
| Particles | Blockage | particles/vial | particles/vial | | particles/vial |
| | Method | ≥25 μm: ≤600 | ≥25 μm: <1 | | ≥25 μm: <1 |
| | | particles/vial | particle/vial | | particle/vial |

### Example 3. Additional Stability Tests of Antibody Formulations

Additional stability quantifications of TB006 antibody formulation drug substance (DS) and drug product (DP) were performed, including at various temperatures, durations, and handling (e.g., freeze thawing).

Table 5 depicts a stability test of a TB006 DS at a 20 mg/mL concentration in a solution of 20 mM L-histidine, 5 mM methionine, 100 mM NaCl, and 0.02% polysorbate-80, pH 5.8. The TB006 DS remained stable under different conditions such as shear stress, freeze thawing, and 40° C. stress test. Diminished binding activity, as determined by biolayer interferometry (BLI), was only observed after prolonged periods of cryogenic storage.

Table 6 depicts a second stability test of a TB006 DS at a 20 mg/mL concentration in a solution of 20 mM L-histidine, 5 mM methionine, 100 mM NaCl, and 0.02% polysorbate-80, pH 5.8. After all conditions, the appearance of the formulations was a colorless, clear liquid. The TB006 DS remained stable under different conditions such as shear stress, freeze thawing, and 40° C. stress test. Diminished binding activity, as determined by BLI, was only observed after prolonged periods of cryogenic storage.

Table 7 depicts a stability test of a TB006 DS at a 50 mg/mL concentration in a solution of 20 mM L-histidine, 5 mM methionine, 100 mM NaCl, and 0.02% polysorbate-80, pH 5.8. After all conditions, the appearance of the formulations was a colorless, clear liquid. The TB006 DS remained stable under different conditions such as shear stress, freeze thawing, and 40° C. stress test. The TB006 antibody in the formulation exhibited stable binding to Gal3 target even after stress conditions as measured by ELISA.

Table 8 depicts a stability test of a TB006 DP at a 20 mg/mL concentration in a solution of 20 mM L-histidine, 5 mM methionine, 100 mM NaCl, and 0.02% polysorbate-80, pH 5.8. The TB006 DP remained stable under a range of temperatures and durations. The DP developed particulate matter only after prolonged periods.

TABLE 5
Stability test of 20 mg/mL TB006 DS

| | | | | | cIEF | cIEF | cIEF | | | |
|---|---|---|---|---|---|---|---|---|---|---|
| | | | | | acidic | main | basic | CE NR | CE R | BLI |
| | | Conc. | | cIEF | peak | peak | peak | monomer | HC + LC | KD |
| | pH | (mg/mL) | SEC | pI | (%) | (%) | (%) | (%) | (%) | (nM) |
| Time 0 (control) | 5.8 | 20.4 | 99.7 | 7.0 | 18.2 | 75.1 | 6.7 | 100.0 | 100.0 | 2.8 |
| Pipetting | 5.9 | 20.5 | 99.7 | 7.0 | 21.9 | 71.0 | 7.1 | 100.0 | 100.0 | 2.8 |
| (shear stress) | | | | | | | | | | |
| Agitation | 5.9 | 20.4 | 99.4 | 7.0 | 17.1 | 75.4 | 7.5 | 100.0 | 100.0 | 2.8 |
| (shear stress) | | | | | | | | | | |
| 3 freeze/thaws | 5.9 | 20.8 | 99.7 | 7.0 | 21.1 | 72.1 | 6.9 | 100.0 | 100.0 | 2.8 |
| 25% vol. of container | | | | | | | | | | |
| 3 freeze/thaws | 5.9 | 20.5 | 99.7 | 7.0 | 22.4 | 70.7 | 6.9 | 100.0 | 100.0 | 2.3 |
| 50% vol. of container | | | | | | | | | | |
| 3 freeze/thaws | 5.9 | 20.6 | 99.7 | 7.0 | 22.0 | 71.1 | 6.9 | 100.0 | 100.0 | 2.6 |
| 75% vol. of container | | | | | | | | | | |
| 3 freeze/thaws | 5.9 | 20.5 | 99.7 | 7.0 | 22.5 | 70.7 | 6.8 | 100.0 | 100.0 | 2.7 |
| 100% vol. of container | | | | | | | | | | |
| 5 freeze/thaws | 5.9 | 20.4 | 99.3 | 7.0 | 17.5 | 74.7 | 7.8 | 100.0 | 100.0 | 2.9 |
| 25% vol. of container | | | | | | | | | | |
| 5 freeze/thaws | 5.9 | 20.5 | 99.2 | 7.0 | 17.9 | 74.5 | 7.6 | 100.0 | 100.0 | 3.1 |
| 50% vol. of container | | | | | | | | | | |
| 5 freeze/thaws | 5.9 | 20.5 | 99.2 | 7.0 | 18.2 | 74.3 | 7.5 | 100.0 | 100.0 | 2.9 |
| 75% vol. of container | | | | | | | | | | |
| 5 freeze/thaws | 5.9 | 20.4 | 99.3 | 7.0 | 18.1 | 74.6 | 7.3 | 100.0 | 100.0 | 2.6 |
| 100% vol. of container | | | | | | | | | | |
| 40° C. for 7 days | 5.9 | 20.2 | 99.7 | 7.0 | 25.4 | 68.1 | 6.6 | 100.0 | 100.0 | 2.4 |
| 40° C. for 14 days | 5.9 | 20.4 | 99.3 | 7.0 | 24.7 | 66.4 | 8.8 | 100.0 | 99.8 | 2.5 |
| 40° C. for 21 days | 5.8 | 20.5 | 99.2 | 7.0 | 29.9 | 61.8 | 8.4 | 100.0 | 99.8 | 2.8 |
| 40° C. for 28 days | 5.9 | 20.5 | 99.3 | 7.0 | 32.6 | 58.7 | 8.7 | 100.0 | 99.7 | 3.1 |
| 25° C. for 14 days | 5.8 | 20.8 | 99.5 | 7.0 | 17.9 | 73.8 | 8.3 | 100.0 | 100.0 | 2.6 |
| 25° C. for 1 month | 5.8 | 20.8 | 99.4 | 7.0 | 19.7 | 72.6 | 7.8 | 100.0 | 100.0 | 3.1 |
| 25° C. for 3 months | 5.7 | 20.9 | 99.4 | 7.0 | 24.2 | 67.9 | 8.0 | 99.7 | 99.8 | 3.3 |
| 25° C. for 6 months | 5.8 | 20.9 | 99.3 | 7.0 | 28.8 | 64.1 | 7.1 | 99.4 | 99.7 | 2.6 |
| 25° C. for 9 months | 5.8 | 20.8 | 99.2 | 7.0 | 33.6 | 59.2 | 7.2 | 98.6 | 99.8 | 2.8 |
| 4° C. for 1 month | 5.9 | 20.5 | 99.3 | 7.0 | 18.1 | 74.3 | 7.6 | 100.0 | 100.0 | 3.3 |
| 4° C. for 3 months | 5.7 | 20.5 | 99.4 | 7.0 | 18.4 | 74.1 | 7.5 | 99.7 | 99.8 | 3.3 |
| 4° C. for 6 months | 5.8 | 20.6 | 99.4 | 7.0 | 16.9 | 76.5 | 6.4 | 99.8 | 99.7 | 2.3 |
| 4° C. for 9 months | 5.8 | 20.6 | 99.4 | 7.0 | 17.4 | 75.9 | 6.7 | 99.7 | 99.9 | 2.2 |
| 4° C. for 12 months | 5.8 | 20.8 | 99.4 | 7.0 | 18.7 | 74.7 | 6.6 | 98.5 | 99.8 | 3.9 |
| 4° C. for 18 months | 5.8 | 20.5 | 99.4 | 7.0 | 18.9 | 74.1 | 6.9 | 99.6 | 99.9 | 2.1 |
| −80° C. for 1 month | 5.9 | 20.5 | 99.4 | 7.0 | 18.6 | 73.4 | 8.0 | 100.0 | 100.0 | 3.2 |
| −80° C. for 3 months | 5.8 | 20.3 | 99.5 | 7.0 | 19.0 | 73.1 | 7.8 | 99.8 | 99.8 | 3.3 |
| −80° C. for 6 months | 5.8 | 20.4 | 99.4 | 7.0 | 16.5 | 77.2 | 6.3 | 99.6 | 99.8 | 2.8 |
| −80° C. for 9 months | 5.9 | 20.5 | 99.4 | 7.0 | 18.0 | 75.8 | 6.2 | 99.8 | 99.9 | 2.0 |
| −80° C. for 12 months | 5.8 | 20.7 | 99.4 | 7.0 | 18.6 | 74.6 | 6.8 | 98.1 | 99.8 | 3.0 |
| −80° C. for 18 months | 5.9 | 19.9 | 99.4 | 7.0 | 19.2 | 72.7 | 8.0 | 99.6 | 99.9 | 2.0 |

TABLE 6
Second stability test of 20 mg/mL TB006 DS

| | | | | | cIEF | cIEF | cIEF | | | |
|---|---|---|---|---|---|---|---|---|---|---|
| | | | | | acidic | main | basic | CE NR | CE R | BLI |
| | | Conc. | | cIEF | peak | peak | peak | monomer | HC + LC | KD |
| | pH | (mg/mL) | SEC | pI | (%) | (%) | (%) | (%) | (%) | (nM) |
| Time 0 (control) | 5.8 | 20.2 | 99.5 | 7.0 | 17.0 | 74.7 | 8.2 | 100.0 | 100.0 | 2.9 |
| Agitation for 1 day | 5.8 | 20.1 | 99.5 | 7.0 | 17.1 | 75.3 | 7.7 | 100.0 | 100.0 | 3.1 |
| (shear stress) | | | | | | | | | | |
| Agitation for 3 days | 5.8 | 20.2 | 99.6 | 7.0 | 17.4 | 74.3 | 8.3 | 100.0 | 100.0 | 3.4 |
| (shear stress) | | | | | | | | | | |
| 3 freeze/thaws | 5.8 | 20.1 | 99.5 | 7.0 | 17.4 | 74.8 | 7.8 | 100.0 | 100.0 | 3.3 |
| 5 freeze/thaws | 5.8 | 20.1 | 99.5 | 7.0 | 18.5 | 73.5 | 8.0 | 100.0 | 100.0 | 3.1 |
| 40° C. for 7 days | 5.7 | 20.4 | 99.6 | 7.0 | 22.2 | 69.0 | 8.9 | 100.0 | 100.0 | 2.9 |
| (forced | | | | | | | | | | |
| degradation) | | | | | | | | | | |
| 40° C. for 14 days | 5.8 | 20.3 | 99.7 | 7.0 | 27.7 | 64.2 | 8.1 | 100.0 | 100.0 | 2.9 |
| (forced | | | | | | | | | | |
| degradation) | | | | | | | | | | |
| 40° C. for 21 days | 5.7 | 20.3 | 99.5 | 7.0 | 29.8 | 61.3 | 8.8 | 100.0 | 100.0 | 2.6 |
| (forced | | | | | | | | | | |
| degradation) | | | | | | | | | | |
| 40° C. for 28 days | 5.8 | 20.1 | 99.4 | 7.0 | 34.7 | 57.4 | 7.9 | 98.5 | 99.8 | 3.8 |
| (forced | | | | | | | | | | |
| degradation) | | | | | | | | | | |
| 25° C. for 14 days | 5.8 | 20.2 | 99.5 | 7.0 | 19.6 | 72.0 | 8.4 | 100.0 | 100.0 | 2.7 |
| (stress test) | | | | | | | | | | |
| 25° C. for 1 month | 5.8 | 20.2 | 99.6 | 7.0 | 19.3 | 73.2 | 7.5 | 99.0 | 100.0 | 3.9 |
| (stress test) | | | | | | | | | | |
| 25° C. for 3 months | 5.75 | 20.4 | 99.5 | 7.0 | 22.1 | 70.3 | 7.6 | 99.6 | 99.5 | 3.4 |
| (stress test) | | | | | | | | | | |
| 25° C. for 6 months | 5.8 | 20.0 | 99.5 | 7.0 | 28.6 | 63.6 | 7.7 | 99.6 | 99.6 | 3.4 |
| (stress test) | | | | | | | | | | |
| 25° C. for 9 months | 5.9 | 20.5 | 99.4 | 7.0 | 33.3 | 60.2 | 6.4 | 98.1 | 98.1 | 2.1 |
| (stress test) | | | | | | | | | | |
| 4° C. for 1 month | 5.9 | 20.2 | 99.6 | 7.0 | 18.7 | 73.7 | 7.6 | 99.1 | 100.0 | 3.7 |
| 4° C. for 3 months | 5.78 | 20.4 | 99.5 | 7.0 | 15.7 | 76.5 | 7.8 | 99.6 | 99.7 | 3.4 |
| 4° C. for 6 months | 5.8 | 20.5 | 99.5 | 7.0 | 18.3 | 74.0 | 7.8 | 99.7 | 99.7 | 3.3 |
| 4° C. for 9 months | 5.9 | 20.6 | 99.4 | 7.0 | 18.0 | 75.4 | 6.6 | 99.2 | 99.0 | 1.9 |
| 4° C. for 12 months | 5.9 | 20.7 | 99.5 | 7.0 | 15.3 | 78.8 | 5.9 | 99.6 | 99.8 | 2.7 |
| −80° C. for 1 month | 5.8 | 20.2 | 99.5 | 7.0 | 18.6 | 74.2 | 7.2 | 99.0 | 100.0 | 3.6 |
| −80° C. for 3 months | 5.79 | 20.5 | 99.5 | 7.0 | 16.9 | 76.6 | 6.5 | 99.6 | 99.6 | 3.5 |
| −80° C. for 6 months | 5.8 | 20.0 | 99.4 | 7.0 | 17.9 | 74.6 | 7.5 | 99.8 | 99.7 | 2.7 |
| −80° C. for 9 months | 5.9 | 20.4 | 99.5 | 7.0 | 17.8 | 75.2 | 6.9 | 99.1 | 98.4 | 1.7 |
| −80° C. for 12 months | 5.9 | 20.3 | 99.5 | 7.0 | 16.7 | 77.3 | 6.0 | 99.6 | 99.7 | 2.7 |
| −80° C. for 18 months | 5.9 | 19.9 | 99.6 | 7.0 | 21.1 | 72.1 | 6.8 | 99.7 | 99.9 | 4.2 |

TABLE 7
Stability test of 50 mg/mL TB006 DS

| | | | | | cIEF | cIEF | cIEF | | | | |
|---|---|---|---|---|---|---|---|---|---|---|---|
| | | | | | acidic | main | basic | CE NR | CE R | BLI | |
| | | Conc. | | cIEF | peak | peak | peak | monomer | HC + LC | KD | ELISA |
| | pH | (mg/mL) | SEC | pI | (%) | (%) | (%) | (%) | (%) | (nM) | potency |
| Time 0 (control) | 5.8 | 49.2 | 99.5 | 7.0 | 16.2 | 77.2 | 6.6 | 98.9 | 100.0 | 2.6 | |
| Agitation for 1 day | 5.9 | 50.3 | 99.5 | 7.0 | 15.1 | 77.7 | 7.2 | 100.0 | 99.7 | 3.8 | |
| (shear stress) | | | | | | | | | | | |
| Agitation for 3 days | 5.8 | 49.8 | 99.5 | 7.0 | 16.8 | 77.3 | 6.5 | 100.0 | 99.7 | 3.7 | |
| (shear stress) | | | | | | | | | | | |
| 3 freeze/thaws | 5.8 | 49.5 | 99.5 | 7.0 | 17.1 | 76.4 | 6.5 | 100.0 | 99.7 | 3.9 | |
| 5 freeze/thaws | 5.8 | 50.0 | 99.5 | 7.0 | 18.2 | 76.3 | 5.5 | 99.8 | 99.7 | 2.6 | |
| 40° C. for 7 days | 5.8 | 49.9 | 99.4 | 7.0 | 20.0 | 72.9 | 7.1 | 100.0 | 98.8 | 2.7 | |
| (forced degradation) | | | | | | | | | | | |
| 40° C. for 14 days | 5.8 | 49.6 | 99.2 | 7.0 | 24.2 | 68.3 | 7.6 | 100.0 | 99.4 | 2.7 | |
| (forced degradation) | | | | | | | | | | | |
| 40° C. for 21 days | 5.8 | 50.0 | 99.1 | 7.0 | 27.6 | 65.0 | 7.4 | 100.0 | 99.7 | 3.0 | |
| (forced degradation) | | | | | | | | | | | |
| 40° C. for 28 days | 5.8 | 50.1 | 99.2 | 7.0 | 34.1 | 58.7 | 7.2 | 98.9 | 99.7 | 2.3 | |
| (forced degradation) | | | | | | | | | | | |
| 40° C. for 3 months | 5.7 | 49.6 | 97.5 | 7.0 | 54.7 | 39.7 | 5.6 | 98.7 | 98.8 | 3.1 | |
| (forced degradation) | | | | | | | | | | | |
| 25° C. for 14 days | 5.8 | 50.2 | 99.5 | 7.0 | 15.0 | 77.9 | 7.1 | 100.0 | 99.4 | 3.1 | |
| (stress test) | | | | | | | | | | | |
| 25° C. for 1 month | 5.8 | 49.7 | 99.5 | 7.0 | 18.4 | 75.0 | 6.6 | 99.8 | 99.8 | 2.2 | |
| (stress test) | | | | | | | | | | | |
| 25° C. for 3 months | 5.8 | 50.7 | 99.3 | 7.0 | 22.3 | 71.0 | 6.6 | 99.7 | 99.6 | 2.5 | |
| (stress test) | | | | | | | | | | | |
| 25° C. for 6 months | 5.8 | 52.1 | 99.3 | 7.0 | 26.3 | 67.0 | 6.8 | 98.7 | 97.8 | 2.4 | 116.0% |
| (stress test) | | | | | | | | | | | |
| 4° C. for 1 month | 5.8 | 50.2 | 99.5 | 7.0 | 17.4 | 76.1 | 6.5 | 99.6 | 99.8 | 2.1 | |
| 4° C. for 3 months | 5.8 | 47.3 | 99.5 | 7.0 | 17.8 | 75.1 | 7.0 | 99.7 | 99.6 | 2.3 | |
| 4° C. for 6 months | 5.8 | 47.9 | 99.5 | 7.0 | 16.8 | 77.3 | 5.9 | 99.1 | 98.1 | 2.1 | 117.0% |
| 4° C. for 9 months | 5.9 | 49.6 | 99.6 | 7.0 | 17.3 | 76.1 | 6.7 | 100.0 | 100.0 | 2.8 | |
| 4° C. for 12 months | 5.8 | 49.6 | 99.6 | 7.0 | 19.5 | 72.9 | 7.6 | 100.0 | 99.7 | 3.7 | |
| −80° C. for 1 month | 5.8 | 50.1 | 99.5 | 7.0 | 16.9 | 76.9 | 6.2 | 99.8 | 99.8 | 2.3 | |
| −80° C. for 3 months | 5.8 | 49.8 | 99.5 | 7.0 | 17.5 | 76.1 | 6.4 | 99.7 | 99.6 | 2.5 | |
| −80° C. for 6 months | 5.9 | 52.2 | 99.5 | 7.0 | 16.1 | 77.6 | 6.2 | 99.0 | 98.3 | 1.8 | 110.3% |
| −80° C. for 9 months | 5.9 | 52.1 | 99.6 | 7.0 | 16.8 | 75.8 | 7.4 | 100.0 | 100.0 | 2.8 | |
| −80° C. for 12 months | 5.8 | 48.3 | 99.6 | 7.0 | 18.4 | 74.0 | 7.5 | 100.0 | 99.9 | 3.5 | |

TABLE 8
Stability test of 20 mg/mL TB006 DP

| | Method | | | | | | | |
|---|---|---|---|---|---|---|---|---|
| | | | | | | | | Non- |
| | | | | | | | | Reduced |
| | cIEF | | | | SEC-UPLC | | | CE-SDS |
| | Acceptance Criteria | | | | | | | |
| | pI: | | | | | | | |
| | 7.0 ± | Acidic | Main | Basic | Mono- | Aggre- | Frag- | |
| | 0.3 | peak: ≤25% | peak: ≥60% | peak: ≤20% | mer: ≥90.0% | gate: ≤10.0% | ment: ≤10.0% | Monomer: ≥90% |
| Time 0 | 7.0 | 18% | 74% | 8% | 99.4% | 0.6% | | 99% |
| (control) | | | | | | | | |
| 5° C. for 1 | 7.0 | 22% | 71% | 7% | 99.4% | 0.6% | | 99% |
| month | | | | | | | | |
| 5° C. for 3 | 7.0 | 19% | 73% | 8% | 99.4% | 0.6% | | 99% |
| months | | | | | | | | |
| 5° C. for 6 | 7.0 | 18% | 74% | 8% | 99.3% | 0.7% | | 99% |
| months | | | | | | | | |
| 5° C. for 9 | 7.0 | 18% | 74% | 8% | 99.4% | 0.6% | | 98% |
| months | | | | | | | | |
| 5° C. for 12 | 7.0 | 17% | 77% | 6% | 99.4% | 0.6% | | 99% |
| months | | | | | | | | |
| 5° C. for 18 | 7.0 | 21% | 72% | 7% | 99.5% | 0.4% | | 99% |
| months | | | | | | | | |
| 25° C. for 1 | 7.0 | 22% | 70% | 8% | 99.4% | 0.6% | | 99% |
| month | | | | | | | | |
| 25° C. for 3 | 7.0 | 23% | 70% | 7% | 99.3% | 0.7% | | 99% |
| months | | | | | | | | |
| 25° C. for 6 | 7.0 | 26% | 66% | 8% | 99.4% | 0.6% | | 99% |
| months | | | | | | | | |

| | Method | | | | | | |
|---|---|---|---|---|---|---|---|
| | | | | | ELISA | | Container |
| | Reduced CE- | Binding | | | Biological | | content |
| | SDS | Activity | Concentration | PS80 | Activity | pH | (USP697) |
| | Acceptance Criteria | | | | | | |
| | Fragment: ≤10% | 50%-150%, | | | 50%-150%, | | Not less |
| | LC + HC: ≥90.0% | kD: 3.0 | 20.0 ± 2 | 0.1-0.3 | IC50: 2.0 | 5.8 ± | than |
| | NGHC: ≤10.0% | nM | mg/mL | mg/ml | μg/mL | 0.5 | 2.0 mL |
| Time 0 | 100.0% | 3.0 nM | 20.3 mg/ml | 0.28 mg/ml | 2.5 μg/mL | 5.8 | 2.2 |
| (control) | | | | | | | |
| 5° C. for 1 | 100.0% | 2.5 nM | 20.0 mg/ml | 0.24 mg/ml | 1.8 μg/mL | 5.8 | 2.2 |
| month | | | | | | | |
| 5° C. for 3 | 100.0% | 3.0 nM | 20.1 mg/ml | 0.21 mg/ml | 1.5 μg/mL | 5.8 | 2.1 |
| months | | | | | | | |
| 5° C. for 6 | 100.0% | 3.5 nM | 20.2 mg/ml | 0.20 mg/ml | 1.5 μg/mL | 5.8 | 2.1 |
| months | | | | | | | |
| 5° C. for 9 | 99.9% | 2.5 nM | 19.9 mg/ml | 0.20 mg/ml | 1.2 μg/mL | 5.8 | 2.1 |
| months | | | | | | | |
| 5° C. for 12 | 99.7% | 3.1 nM | 20.0 mg/ml | 0.24 mg/ml | 1.5 μg/mL | 5.9 | 2.0 |
| months | | | | | | | |
| 5° C. for 18 | 99.1% | 2.0 nM | 20.6 mg/ml | | 1.8 μg/mL | 5.8 | |
| months | | | | | | | |
| 25° C. for 1 | 100.0% | 2.5 nM | 19.9 mg/ml | 0.24 mg/ml | 1.8 μg/mL | 5.9 | 2.1 |
| month | | | | | | | |
| 25° C. for 3 | 100.0% | 3.1 nM | 20.1 mg/ml | 0.22 mg/ml | 1.5 μg/mL | 5.9 | 2.2 |
| months | | | | | | | |
| 25° C. for 6 | 100.0% | 3.7 nM | 20.2 mg/ml | 0.19 mg/ml | 1.5 μg/mL | 5.8 | 2.2 |
| months | | | | | | | |

| | Method | | | | | | |
|---|---|---|---|---|---|---|---|
| | | | | | | | Container |
| | | | | | | | Closure |
| | | | | | | | Integrity |
| | | | | Bacterial | | | Test (5° C.) |
| | | Osmolality | Sterility | Endotoxins | Particulate Matter | | (Selblty-28 |
| | Appearance | (USP785) | (USP71) | (USP85) | (USP788) | | MOD 5° C.) |
| | Acceptance Criteria | | | | | | |
| | Clear, colorless to | | Complies | | | | |
| | light yellow, | | with the | | ≤6000 Par- | ≤600 Par- | |
| | essentially free of | 230 ± 25 | test for | ≤0.6 | ticles/vial | ticles/vial | </=5.7E−06 |
| | particles | mOsm/kg | sterility | EU/mg | for ≥10 μm | for ≥25 μm | mbar*L/s |
| Time 0 | Clear, colorless, | 228 mOsm/ | Pass | <0.01 | 9.0 | <1 | N/A |
| (control) | essentially free of | kg | | EU/mg | | | |
| | particles | | | | | | |
| 5° C. for 1 | Clear, colorless, | 226 mOsm/ | N/A | N/A | N/A | | N/A |
| month | essentially free of | kg | | | | | |
| | particles | | | | | | |
| 5° C. for 3 | Clear, colorless, | 227 mOsm/ | N/A | N/A | 6.0 | <1 | N/A |
| months | essentially free of | kg | | | | | |
| | particles | | | | | | |
| 5° C. for 6 | Clear, colorless, | 227 mOsm/ | Pass | N/A | 13.0 | <1 | Pass |
| months | essentially free of | kg | | | | | |
| | particles | | | | | | |
| 5° C. for 9 | Clear, colorless, | 229 mOsm/ | N/A | N/A | 15.0 | <1 | N/A |
| months | essentially free of | kg | | | | | |
| | particles | | | | | | |
| 5° C. for 12 | Clear, colorless, | 226 mOsm/ | Pass | <0.01 | 59.0 | 1.0 | Pass |
| months | essentially free of | kg | | EU/mg | | | |
| | particles | | | | | | |
| 5° C. for 18 | | | | | | | |
| months | | | | | | | |
| 25° C. for 1 | Clear, colorless, | 229 mOsm/ | N/A | N/A | N/A | | |
| month | essentially free of | kg | | | | | |
| | particles | | | | | | |
| 25° C. for 3 | Clear, colorless, | 231 mOsm/ | N/A | N/A | 7 | <1 | |
| months | essentially free of | kg | | | | | |
| | particles | | | | | | |
| 25° C. for 6 | Clear, colorless, | 229 mOsm/ | N/A | N/A | 14 | 1 | |
| months | essentially free of | kg | | | | | |
| | particles | | | | | | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A pharmaceutical antibody formulation comprising:
a therapeutically effective amount of an antibody, wherein the antibody comprises a heavy chain CDR1 (HCDR1) having the sequence of SEQ ID NO: 2, a heavy chain CDR2 (HCDR2) having the sequence of SEQ ID NO: 3, a heavy chain CDR3 (HCDR3) having the sequence of SEQ ID NO: 4, a light chain CDR1 (LCDR1) having the sequence of SEQ ID NO: 5, a light chain CDR2 (LCDR2) having the sequence of SEQ ID NO: 6; and a light chain CDR3 (LCDR3) having the sequence of SEQ ID NO: 7;
histidine;
methionine;
NaCl; and
polysorbate, wherein the formulation is at a pH between 5.3 and 6.3.

2. The pharmaceutical antibody formulation of claim 1, wherein the histidine is L-histidine, and wherein the L-histidine is present at 10 to 50 mM.

3-4. (canceled)

5. The pharmaceutical antibody formulation of claim 1, wherein the methionine is present at 2 to 10 mM.

6. (canceled)

7. The pharmaceutical antibody formulation of claim 1, wherein the NaCl is present at 50 to 150 mM.

8. (canceled)

9. The pharmaceutical antibody formulation of claim 1, wherein the polysorbate comprises polysorbate-20, polysorbate-40, polysorbate-60, polysorbate-80, or any combination thereof.

10. (canceled)

11. The pharmaceutical antibody formulation of claim 9, wherein the polysorbate comprises polysorbate 80, wherein the polysorbate 80 is present at 0.01 to 0.04%.

12. (canceled)

13. The pharmaceutical antibody formulation of claim 1, wherein the pH is about 5.8.

14. (canceled)

15. The pharmaceutical antibody formulation of claim 1, further comprising sucrose, mannitol, or both, wherein the sucrose and/or mannitol is present at 2% to 5% or about 2% to about 5%.

16-17. (canceled)

18. The pharmaceutical antibody formulation of claim 1, wherein the antibody is present at an amount of 70 to 7500 mg as a unit dose.

19-21. (canceled)

22. The pharmaceutical antibody formulation of claim 1, wherein the antibody is present at a concentration of one of: 1 mg/mL, 5 mg/mL, 10 mg/mL, 20 mg/mL, 40 mg/mL, or 50 mg/mL, or any concentration within a range defined by any two of the aforementioned concentrations.

23. The pharmaceutical antibody formulation of claim 1, wherein L-histidine is present at about 20 mM, methionine is present at about 5 mM, NaCl is present at about 100 mM, polysorbate 80 is present at about 0.02%, sucrose is present at 2-5%, mannitol is present at 2-5%, the pH is about 5.8, and wherein the therapeutically effective amount of the antibody is one of: 70 mg, 75 mg, 140 mg, 200 mg, 420 mg, 450 mg, 700 mg, 1500 mg, 2100 mg, 3750 mg, 5000 mg, or 7500 mg as a unit dose, or any amount within a range defined by any two of the aforementioned amounts.

24-25. (canceled)

26. A pharmaceutical antibody formulation comprising:
a therapeutically effective amount of an antibody,
wherein the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, and wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg;
L-histidine is present at 20 mM;
methionine is present at 5 mM;
NaCl is present at 100 mM;
polysorbate 80 is present at 0.02%; and
the pH is about 5.8.

27. The pharmaceutical antibody formulation of claim 26, wherein the antibody is present at an amount as a unit dose of: 70 mg, 75 mg, 450 mg, 1500 mg, 3750 mg, or 7500 mg.

28. (canceled)

29. The pharmaceutical antibody formulation of claim 26, wherein sucrose is present at 2-5% and mannitol is present at 2-5%.

30-34. (canceled)

35. The pharmaceutical antibody formulation of claim 26, wherein the pharmaceutical antibody formulation is prepared at a concentration of antibody of 20 mg/mL or 50 mg/mL.

36. (canceled)

37. A pharmaceutical antibody formulation comprising:
a therapeutically effective amount of an antibody, wherein the antibody comprises a HCDR1 having the sequence of SEQ ID NO: 2, a HCDR2 having the sequence of SEQ ID NO: 3, a HCDR3 having the sequence of SEQ ID NO: 4, a LCDR1 having the sequence of SEQ ID NO: 5, a LCDR2 having the sequence of SEQ ID NO: 6; and a LCDR3 having the sequence of SEQ ID NO: 7, wherein each CDR can have up to 1, 2, 3, 4, or 5 amino acids changed from the recited sequence;
histidine;
methionine;
NaCl; and
polysorbate, wherein the formulation is at a pH between 5.3 and 6.3.

38. The pharmaceutical antibody formulation of claim 37, further comprising sucrose or mannitol, or both.

39. The pharmaceutical antibody formulation of claim 37, wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg.

40-41. (canceled)

42. A sterile vial comprising a pharmaceutical antibody formulation, wherein the pharmaceutical antibody formulation comprises a therapeutically effective amount of an antibody, wherein the antibody comprises an HCDR1 having the sequence of SEQ ID NO; 2, an HCDR2 having the sequence of SEQ ID NO: 3, an HCDR3 having the sequence of SEQ ID NO; 4, an LCDR1 having the sequence of SEQ ID NO: 5, an LCDR2 having the sequence of SEQ ID NO: 6; and an LCDR3 having the sequence of SEQ ID NO: 7.

43. The sterile vial of claim 42, wherein the pharmaceutical antibody formation further comprises histidine, methionine, NaCl, and polysorbate, and wherein the formulation is at a pH between 5.3 and 6.3.

44. (canceled)

45. The sterile vial of claim 42, wherein the sterile vial contains 2, 3, 4, 5, 6, 7, 8, 9, or 10 mL of the pharmaceutical antibody formulation.

46-47. (canceled)

48. The sterile vial of claim 42, wherein the pharmaceutical antibody formulation is a concentrated form of the pharmaceutical antibody formulation of claim 1.

49. The sterile vial of claim 48, wherein the concentrated form of the pharmaceutical antibody formulation is at a concentration of 20, 30, 40, 50, 60, 70, 80, 90, or 100 mg/mL of antibody, or any concentration within a range defined by any two of the aforementioned concentrations.

50-51. (canceled)

52. The sterile vial of claim 48, wherein the concentrated form of the pharmaceutical antibody formulation is intended to be diluted 1×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, or 100× fold, or any fold within a range defined by any two of the aforementioned fold.

53. The sterile vial of claim 48, wherein the concentrated form of the pharmaceutical antibody formulation is intended to be diluted to 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations.

54. The sterile vial of claim 48, wherein the concentrated form of the pharmaceutical antibody formulation is intended to be diluted into a final volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes.

55-61. (canceled)

62. The pharmaceutical antibody formulation of claim 1, wherein the antibody comprises a heavy chain variable domain (VH) region having a sequence at least 80%, 85%, 90%, 95%, 99%, or 100% identical to that of SEQ ID NO: 8.

63. The pharmaceutical antibody formulation of claim 1, wherein the antibody comprises a light chain variable domain (VL) region having a sequence at least 80%, 85%, 90%, 95%, 99%, or 100% identical to that of SEQ ID NO: 9.

64. The pharmaceutical antibody formulation of claim 1, wherein the antibody comprises a VH region having a sequence at least 80%, 85%, 90%, 95%, 99%, or 100% identical to that of SEQ ID NO: 8, and wherein the antibody comprises a VL region having a sequence at least 80%, 85%, 90%, 95%, 99%, or 100% identical to that of SEQ ID NO: 9.

65-67. (canceled)

68. The pharmaceutical antibody formulation of claim 1, wherein the antibody comprises a heavy chain (HC) having a sequence at least 80%, 85%, 90%, 95%, 99%, or 100% identical to that of SEQ ID NO: 10.

69. The pharmaceutical antibody formulation of claim 1, wherein the antibody comprises a light chain (LC) having a sequence at least 80%, 85%, 90%, 95%, 99%, or 100% identical to that of SEQ ID NO: 11.

70-71. (canceled)

72. The pharmaceutical antibody formulation of claim 1, wherein the antibody comprises a VH that is encoded by a nucleic acid sequence having at least 80%, 85%, 90%, 95%, 99%, or 100% identical to that of SEQ ID NO: 12.

73. The pharmaceutical antibody formulation of claim 1, wherein the antibody comprises a VL that is encoded by a nucleic acid sequence having at least 80%, 85%, 90%, 95%, 99%, or 100% identical to that of SEQ ID NO: 13.

74-75. (canceled)

76. The pharmaceutical antibody formulation of claim 1, wherein the antibody comprises an HC that is encoded by a nucleic acid sequence having at least 80%, 85%, 90%, 95%, 99%, or 100% identical to that of SEQ ID NO: 14.

77. The pharmaceutical antibody formulation of claim 1, wherein the antibody comprises an LC that is encoded by a nucleic acid sequence having at least 80%, 85%, 90%, 95%, 99%, or 100% identical to that of SEQ ID NO: 15.

78-87. (canceled)

88. The pharmaceutical antibody formulation of claim 1, wherein the formulation exhibits a dissociation constant (KD) of 1.7-4.2 or about 1.7-4.2 as determined by biolayer interferometry (BLI) when stored at a) 40° C. for 7, 14, 21, or 28 days, b) 25° C. for 14 days or 1, 3, 6, or 9 months, c) 4° C. for 1, 3, 6, 9, 12, or 18 months, or d) −80° C. for 1, 3, 6, 9, 12, or 18 months, and/or after being subjected to shear stress or freeze thaws, optionally 3 or 5 freeze thaws.

89-101. (canceled)

102. A method of treating Alzheimer's disease, the method comprising:
administering a pharmaceutical antibody formulation to a subject in need of Alzheimer's disease treatment, wherein the pharmaceutical antibody formulation comprises:
a therapeutically effective amount of the antibody,
wherein the antibody comprises an HCDR1 having the sequence of SEQ ID NO: 2, an HCDR2 having the sequence of SEQ ID NO: 3, an HCDR3 having the sequence of SEQ ID NO: 4, an LCDR1 having the sequence of SEQ ID NO: 5, an LCDR2 having the sequence of SEQ ID NO: 6; and an LCDR3 having the sequence of SEQ ID NO: 7, and wherein the antibody is present at an amount as a unit dose of: 70 mg, 75 mg, 140 mg, 200 mg, 420 mg, 450 mg, 700 mg, 1500 mg, 2100 mg, 3750 mg, 5000 mg, or 7500 mg;
L-histidine is present at 20 mM;
methionine is present at 5 mM;
NaCl is present at 100 mM;
polysorbate 80 is present at 0.02%; and
the pH is about 5.8.

103. (canceled)

104. The method of claim 102, wherein the subject is administered 70 mg, 75 mg, 140 mg, 200 mg, 420 mg, 450 mg, 700 mg, 1500 mg, 2100 mg, 3750 mg, 5000 mg, or 7500 mg of antibody as a unit dose, or any amount of antibody as a unit dose within a range defined by any two of the aforementioned amounts.

105-107. (canceled)

108. The method of claim 102, wherein the unit dose is administered over the course of 10, 20, 30, 40, 50, 60, 70, 80, 90, 100, 120, 130, 140, 150, 160, 170, 180, 190, or 200 minutes.

109. (canceled)

110. The method of claim 102, wherein the pharmaceutical antibody formulation is first diluted prior to administration such that the antibody is at a concentration of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/mL or any concentration within a range defined by any two of the aforementioned concentrations.

111. The method of claim 102, wherein the pharmaceutical antibody formulation is first diluted prior to administration such that the pharmaceutical antibody formulation is administered in a volume of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mL, or any volume within a range defined by any two of the aforementioned volumes.

112-121. (canceled)
